(19) United States
(12) Patent Application Publication
(10) Pub. No.: US 2025/0222170 A1
(43) Pub. Date: Jul. 10, 2025

(54) MODIFIED BIOLOGICAL BONE MINERAL SCAFFOLD DOPED BASED ON LITHIUM MAGNESIUM PHOSPHATE

(71) Applicant: Ningbo Cibei Medical Treatment Appliance Co., Ltd., Cixi (CN)

(72) Inventors: Linyang CHU, Cixi (CN); Fei ZHANG, Cixi (CN); Yaping LI, Cixi (CN); Guofeng ZHANG, Cixi (CN); Tianwei SHEN, Cixi (CN); Jiamin HUANG, Cixi (CN); Bin SUN, Cixi (CN); Qiongwei CHEN, Cixi (CN); Jiuhan LI, Cixi (CN); Xiaosong LI, Cixi (CN); Yinchen ZHU, Cixi (CN); Minghao ZHENG, Cixi (CN); Jie HUANG, Cixi (CN)

(73) Assignee: Ningbo Cibei Medical Treatment Appliance Co., Ltd., Cixi (CN)

(21) Appl. No.: 19/023,849

(22) Filed: Jan. 16, 2025

Related U.S. Application Data
(63) Continuation of application No. PCT/CN2024/072928, filed on Jan. 18, 2024.

(30) Foreign Application Priority Data
Jan. 4, 2024 (CN) 202410011324.9

(51) Int. Cl. A61L 27/36; A61L 27/04; A61L 27/12

(52) U.S. Cl. CPC A61L 27/3608; A61L 27/042; A61L 27/047; A61L 27/12; A61L 27/365; A61L 27/3687; A61L 27/3691; A61L 2300/112; A61L 2300/412; A61L 2300/604; A61L 2300/63; A61L 2400/18; A61L 2430/02; A61L 2430/40

(57) ABSTRACT

The present invention discloses a modified biological bone mineral scaffold doped based on lithium magnesium phosphate, which is obtained by immersing a calcined bovine or porcine cancellous bone mineral porous scaffold into a solution containing one or more of active metal ions of magnesium, lithium, strontium, zinc, iron and calcium and a phosphorus source composite solution for hydrothermal reaction, baking, drying and calcining at a high temperature. The modified biological bone mineral scaffold doped based on lithium magnesium phosphate obtained in the present invention can effectively stabilize the doped magnesium lithium phosphate and one or more of other osteogenic active ions of strontium, zinc, iron and calcium. The material maintains the three-dimensional interconnected mesh structure and natural crystal structure of the calcined bovine or porcine cancellous bone mineral porous scaffold, and has calcium phosphate crystals containing active ions.

### CROSS-REFERENCE TO RELATED APPLICATIONS

The application claims priority to Chinese patent application No. 202410011324.9, filed on Jan. 4, 2024, the entire contents of which are incorporated herein by reference.

### TECHNICAL FIELD

The present invention relates to the technical field of materials for bone transplantation, and in particular to a modified biological bone mineral scaffold doped based on lithium magnesium phosphate.

### BACKGROUND

Bone transplantation is common tissue transplantation second only to blood transfusion. Autogenous bone graft is the gold standard for bone defect repair, because the three elements of the autogenous bone repair include cells, active proteins and mineralized extracellular matrix scaffolds (including hydroxyapatite as main component, and other ions such as Mg, Fe, Si, Zn, Mn, Cu, Na, K, F, S, C and Cl). The research and development of substitute materials for bone transplantation is one of the focuses of medical research at present. There are extensive homogeneous substitution behaviors in the process of human home mineralization, and human bone is complex in composition and structure. In the design of substitute materials for bone transplantation (artificial bone) or scaffolds for bone tissue engineering, it is crucial to consider the complex composition and structure (unique three-dimensional interconnected mesh structure and natural nanocrystalline structure) and physical and chemical properties (e.g., good hydrophilicity, degradability, mechanical strength, etc.) of the severely mineralized tissue of human bone.

Human bone cannot be completely replaced by limited characteristics provided by a single material. More importantly, the ideal artificial bone or scaffold for bone tissue engineering must provide a three-dimension porous microstructure for regeneration of the bone tissue and provide an empty space for adhesion and physiological activities of osteogenesis related cells, and the scaffold must have components similar to human hone mineral. Just like the dissolution of the autogenous bone mineralized extracellular matrix scaffold, the dissolution of the scaffold [in terms of physical, chemical and cellular aspects] can provide calcium phosphate ions and other osteogenic active ions such as magnesium ions, forming a microenvironment with high-calcium osteognic active ions.

Calcium phosphate is the main component of human bone mineral, and the calcium phosphate material is also the most common substitute material for bone transplantation in clinic at present. At present, there is a lack of ideal substitute materials for bone transplantation in clinic, mainly manifested in the ideal three-dimensional interconnected mesh structure, and high porosity and specific surface area, appropriate degradability, good hydrophilicity, osteoconduction, osteoinductivity, mechanical strength and other ideal properties cannot be achieved concurrently.

The ideal three-dimensional interconnected mesh structure is the most basis requirement for the scaffold for bone tissue engineering or the substitute material for bone transplantation. The artificial bone with a relatively ideal three-dimensional interconnected mesh microstructure that has been applied in clinic is derived from animal materials. The porous hydroxyapatite ceramic bone (including Geistlich artificial bone sold best in China) prepared from bovine cancellous bone through a high temperature sintering procedure is characterized in that its composition, structure and pore diameter are highly similar to those of the human cancellous bone mineralized extracellular matrix, and has good biocompatibility, good osteoconduction, high mechanical strength and the like. After being implanted into a body, it is beneficial to the recruitment of bone repair cells, the adhesion of active factors, the exchange between oxygen and tissue fluid and the formation and entry of blood vessels, and provides good adhesion support and physiological activity space for bone repair cells. However, its big disadvantage is that the bone mineral (i.e., “elementary” hydroxyapatite) obtained by sintering bovine cancellous bone at a high temperature is too stable, degrades slowly in vivo, has the lowest solubility among calcium phosphate materials for bone transplantation and cannot release high-concentration calcium phosphate and other osteogenic beneficial ions. As a result, it lacks high osteogenic activity, and it is not conducive to the repair and transformation of bone.

The ideal degradation speed is another important requirement for the artificial bone or the scaffold for bone tissue engineering. The ideal degradation speed of the artificial bone should be matched with the formation speed of new bone, and the artificial bone gradually degrades to provide space for new bone substitution when guiding the formation of new bone. The osteogenic beneficial ions such as calcium ions released continuously in the degradation process provide mineral recombination components for the re-deposition, transformation and metabolism of bone mineral. This process may stimulate the formation of new bone, that is, it has a certain degree of potential bone induction, which is the biochemical basis for the osteogenic activity of mineral materials. A too high degradation speed of the artificial bone or the scaffold for bone tissue engineering is not conducive to providing sufficient space-time support and guidance for the bone repair process, while a too low degradation speed will hinder the formation, substitution and modeling of new bone. The in-vitro degradation of a mineral material for bone transplantation is related to the composition of the material, as well as the particle size, porosity, specific surface area, crystallinity and solubility of the material, among which solubility is an important factor. In recent 20 years, some scientists have tried to convert calcined bovine cancellous bone porous hydroxyapatite (the molar ratio of calcium to phosphorus is 5:3) doped phosphorus into tricalcium phosphate (the molar ratio of calcium to phosphorus is 3:2), calcium pyrophosphate (the molar ratio of calcium to phosphorus is 1:1) or a multiphase calcium phosphate porous scaffold containing tricalcium phosphate, calcium pyrophosphate and hydroxyapatite in order to improve the degradation characteristics and osteogenic activity of the material.

In recent ten years, there has been another hotspot in the research of artificial bone: the research on the influence of the doping of some metal ions on osteogenic activity. There is a summary that some metal ions promote the proliferation of stem cells, osteogenic differentiation, physiological activities and differentiation of osteoblasts, bone formation and mineralization, and inhibit major links of bone formation and repair, such as osteoclast differentiation and vascular endothelial cells, so as to regulate bone formation. Lithium, silver and aluminum ions promote the proliferation of stem cells; copper ions inhibit the proliferation of stem cells; silver, calcium, lithium, magnesium, vanadium and zinc ions promote osteogenic differentiation; aluminum, cobalt, copper, iron and manganese ions inhibit osteogenic differentiation; silver, calcium, gallium, lithium, magnesium and vanadium ions promote the physiological activities and differentiation of osteoblasts; aluminum, cobalt, copper, iron and manganese ions inhibit the physiological activities and differentiation of osteoblasts; silver, calcium, cobalt, copper, lithium, magnesium, strontium and zinc ions promote bone formation; aluminum and iron ions inhibit bone formation; silver, calcium, lithium, magnesium, manganese, vanadium and zinc ions promote mineralization; aluminum, copper, cobalt and iron ions inhibit mineralization; lithium, gallium, zinc and strontium ions inhibit osteoclast differentiation; iron ions promote osteoclast differentiation; cobalt, copper, manganese and gallium ions promote the proliferation and revascularization of endothelial cells; and, silver, copper, gallium and zinc ions inhibit biological membranes.

As the most common mineral substance, calcium is mainly stored in bones. Calcium homeostasis is closely regulated by parathormone (PTH) and calcitonin, and the serum calcium level of osteoclasts is regulated by stimulating (PTH) or inhibiting (calcitonin) mediated bone absorption. In the bone remodeling process, data have showed that bone absorption osteoclasts will produce a local concentration of extracellular calcium ions up to 40 mol/L. The calcium increase in these microenvironments inhibits the absorption activity of osteoclasts and promotes the proliferation and differentiation of mesenchymal cells into stromal cells and osteoblasts. In 1980s, it was proved that extracellular calcium could activate extracellular calcimedin-coupled receptors, which were called calcium sensor receptors (CaSRs). Regarding the high reactivity of bone cells to extracellular calcium, the increase in the calcium level can promote the proliferation, chemotaxis and psteogenic differentiation of bone cells. By activating CaSRs, mesenchymal stromal cells derived from bone marrow can be activated in a dose-dependent manner. Tricalcium phosphate (the molar ratio of calcium to phosphorus is 3:2) and calcium pyrophosphate (the molar ratio of calcium to phosphorus is 1:1) have better degradability than hydroxyapatite (the molar ratio of calcium to phosphorus is 5:3), and artificial materials containing tricalcium phosphate and calcium pyrophosphate are more beneficial than those containing hydroxyapatite to the formation of a microenvironment with a higher concentration of calcium ions in the transplanted part. Magnesium ions, as divalent cations hotly researched at present, are active ions that have been applied in clinic. Magnesium exists widely in the nature world, and about 25 g magnesium is contained in a human adult body. Magnesium plays an important role in the formation of human bone and all growth processes, maintaining the structure and function of bones, bone metabolism and remodeling. Magnesium calcium phosphate-based bone cement with a low magnesium content can significantly improve the adhesion ability of cells. The magnesium-doped calcium phosphate bone cement has become an increasingly valued novel biomaterial for bone repair because it can promote the formation of a bone interface between the implant material and the bone tissue. The magnesium-doped bone cement is easy to prepare, and the bone cement with 73% b-tricalcium phosphate/21% calcium dihydrogen phosphate/5% magnesium hydrogen phosphate has been used in clinic in Western countries such as New Zealand. The compound magnesium-containing bone cement is degradable, can release calcium, phosphorus, magnesium and other elements that are beneficial to bone formation and can be degraded and ion exchanged in the body after transplantation, but has no three-dimensional interconnected mesh structure and thus prevents from repaired cells and blood vessels from penetrating into the transplanted object at the early stage. Magnesium ions promote osteogenic differentiation, physiological activities and differentiation of osteoblasts, bone formation and mineralization. Out previous research suggested that magnesium ion-doped modified biological bone mineral scaffold could improve osteogenesis and vascularization.

As a trace element indispensable for the human body, lithium does not have any known function for human organism. However, due to its beneficial effects on psychotherapy, lithium has been widely introduced into medical applications. Various mechanisms of lithium action have been proposed, and it is widely recognized that stimulating the proliferation of neural progenitor cells through a Wnt/β-catenin pathway leads to the increase of gray matter in the brain. Interestingly, the proliferation of other types of cells, such as mesenchymal stem cells, is also regulated by the Wnt/β-catenin pathway, indicating that lithium may also regulate the proliferation of cells. In fact, a recent research reported that lithium-mediated Wnt/β-catenin signals stimulated the proliferation of hMSCs. In addition, the previous research indicates that lithium in this pathway is the main regulatory factor for osteoblast production, and its application in tissue engineering is more fascinating. In rats receiving lithium solution through gastric administration and rats receiving lithium salt solution, the increase of the bone mineral density, the number of new mature bone tissues and the bone mass regeneration indicates that lithium can accelerate callus ossification and bone healing. The preliminary experiments of lithium release, toxicity and osteoblast activity of the lithium-doped bone cement suggest that lithium is a promising metal ion, and the in-vivo application of the lithium-doped bone cement significantly improves the bone formation rate and defect repair rate and has better osteoconduction than the pure calcium phosphate bone cement. It seems that lithium can directly regulate and promote the proliferation of stem cells, osteogenic differentiation, physiological activities and differentiation of osteoblasts, bone formation and mineralization and inhibit major links of bone formation and repair, such as osteoclast differentiation, so as to facilitate bone formation.

Silver can promote the proliferation of stem cells, osteogenic differentiation, physiological activities and differentiation of osteoblasts, bone formation and mineralization, and inhibit biological membranes, so as to facilitate bone formation. However, since it is difficult to consider the harm of silver to the human body up to now, silver cannot be used as a doping element. For the same reason, copper, manganese, cobalt and aluminum are not selected as doping elements.

In the early stage, we made efforts to effectively dope two or more of osteogenic active ions, such as magnesium, magnesium zinc, magnesium strontium, phosphate radical and sulfate radical, into the porous hydroxyapatite bone mineral scaffold of calcined bovine bone, and also tried to improve the degradability and osteogenic activity of the calcined bovine hydroxyapatite while maintaining its ideal three-dimensional interconnected mesh structure and good mechanical strength.

### SUMMARY

The objective of the present invention is to solve the above problem and provide a modified biological bone mineral scaffold doped based on lithium magnesium phosphate so as to obtain a scaffold for bone tissue engineering and a substitute material for bone transplantation with good three-dimensional interconnected mesh structure, mechanism strength, degradability and osteogenic activity.

To achieve the above objective, the present invention provides a modified biological bone mineral scaffold doped based on lithium magnesium phosphate, which is obtained by immersing a calcined bovine or porcine cancellous bone mineral porous scaffold into a solution containing one or more of active metal ions of magnesium, lithium, strontium, zinc, iron and calcium and a phosphorus source composite solution for hydrothermal reaction, baking, drying and calcining at a high temperature. Here, the metal ion calcium refers to supplementary calcium, and does not contain calcium inherent in the biological bone mineral scaffold.

An alternative for the immersing a calcined bovine or porcine cancellous bone mineral porous scaffold into a solution containing one or more of active metal ions of magnesium, lithium, strontium, zinc, iron and calcium and a phosphorus source composite solution for hydrothermal reaction is: immersing the calcined bovine or porcine cancellous bone mineral porous scaffold into a metal ion source solution containing one or more of active metal ions of magnesium, lithium, strontium, zinc, iron and calcium and a white granulated sugar solution, drying off the liquid by microwave or a constant temperature box, baking and drying at 96° C. to 198° C., and then putting into the phosphorus source composite solution for hydrothermal reaction, the phosphorus source composite solution being a phosphorus source binary system so as to form a phosphorus source binary system containing osteogenic active metal ions.

The hydrothermal reaction is in a constant temperature hydrothermal mode, controlled at 60° C. to 100° C. for 24 to 48 hours.

In the modified biological bone mineral scaffold doped based on lithium magnesium phosphate provided by the present invention, the material-to-liquid ratio of the calcined bovine or porcine cancellous bone mineral porous scaffold to the metal ion source solution may be 15-50 g: 100 mL, and the material-to-liquid ratio of the calcined bovine or porcine cancellous bone mineral porous scaffold to the phosphorus source composite solution may be 15-50 g: 100 mL.

In the metal ion source solution, the magnesium source is one of magnesium acetate, magnesium sulfate and magnesium hydrogen phosphate, etc.; the lithium source is lithium chloride; the calcium source is one of calcium chloride and calcium hydroxide; the zinc source is one of soluble zinc salts, such as zinc nitrate or zinc acetate; the strontium source is one of soluble strontium salts, such as strontium nitrate, strontium acetate or strontium sulfate; the iron source is one of soluble iron salts, such as ferrous sulfate, ferrous chloride, ferric trichloride or ferric acetate; the phosphorus source binary system is phosphoric acid and a soluble phosphate composite solution; and, the soluble phosphate composite solution is one or a combination of diammonium hydrogen phosphate, ammonium dihydrogen phosphate, disodium hydrogen phosphate, sodium dihydrogen phosphate, dipotassium hydrogen phosphate, potassium dihydrogen phosphate and magnesium hydrogen phosphate.

The final concentration of magnesium ions in the phosphorus source binary system containing osteogenic active metal ions is 0.05 to 0.20 mol/L; the final concentration of lithium ions in the phosphorus source binary system containing osteogenic active metal ions is 0.06 to 0.6 mol/L; the final concentration of zinc ions in the phosphorus source binary system containing osteogenic active metal ions is 0.1 to 0.6 mol/L; the final concentration of strontium ions in the phosphorus source binary system containing osteogenic active metal ions is 0.15 to 0.9 mol/L; the final concentration of ferrous ions in the phosphorus source binary system containing osteogenic active metal ions is 0.1 to 0.6 mol/L; the final concentration of supplementary calcium ions in the phosphorus source binary system containing osteogenic active metal ions is 0.15 to 1.5 mol/L; preferably, the final concentration of phosphate radials provided by phosphoric acid in the phosphorus source binary system containing osteogenic active metal ions is 0.15 to 0.9 mol/L; the final concentration of phosphate radials provided by soluble phosphate in the phosphorus source binary system containing osteogenic active metal ions is 0.06 to 0.6 mol/L; and, preferably, the ratio of the total molar concentration of osteogenic active cations including calcium in the reaction system (the molar number of monovalent lithium ions is halved, and the molar concentration of trivalent iron ions is multiplied by 1.5) to the molar concentration of phosphorus ions is 1.1-1.6:1.

After hydrothermal reaction, the liquid is dried off at a constant temperature, and baking and drying are preferably performed at 75° C. to 198° C.

During the high-temperature calcination, the high-temperature calcination is performed at 750° C. to 1200° C. for 6 to 24 hours.

In the modified biological bone mineral scaffold doped based on lithium magnesium phosphate provided by the present invention, a method for preparing the calcined bovine or porcine cancellous bone mineral porous scaffold is as follows:
(1): strip or block-shaped calcined bovine or porcine cancellous bone mineral porous scaffold
(1-1): cutting a bovine or porcine cancellous bone into cancellous bone strips or blocks with a thickness of 0.5 to 5 cm to obtain a raw material bone, where it is also possible to prepare a cylindrical calcellous bone strip by trephination;
(1-2): stewing the raw material bone for 36 to 60 min in distilled water in a pressure cooker, then cleaning with drinking water at 50° C. to 75° C., and repeating this step for 5 to 6 times; and
(1-3): drying the raw material bone treated in the step (1-2) for 12 to 24 hours in a constant temperature box at 80° C. to 120° C., then calcining the raw material bone for 6 to 12 hours in a calcination furnace at 900° C. to 1200° C., and then slowly cooling to the room temperature to obtain calcined cancellous bone mineral porous scaffolds, the powder diffraction components of the bovine or porcine cancellous bone mineral material being hydroxyapatite;
(2): particulate bovine or porcine cancellous bone mineral porous scaffold
(2-1): cutting bovine or porcine cancellous bone into bone strips or bone blocks with a thickness of 0.5 to 6 cm to obtain a raw material bone;
(2-2): stewing the raw material bone for 36 to 60 min in distilled water in a pressure cooker, then cleaning with drinking water at 50° C. to 75° C., and repeating this step for 5 to 6 times;
(2-3): drying the raw material bone treated in the step (2-2) for 12 to 24 hours in a constant temperature box at 80° C. to 120° C., then calcining the raw material bone for 6 to 12 hours in a calcination furnace at 900° C. to 1200° C., and then slowly cooling to the room temperature to obtain calcined bovine or porcine cancellous bone mineral porous scaffolds; and
(2-4): crushing the bovine cancellous bone mineral porous scaffolds treated in the step (2-3) by a food crushing machine, and screening particulate bovine cancellous bone mineral porous scaffolds of various specifications such as 0.2-1 mm, 1-3 mm, 3-5 mm and 5-7 mm for further use by a stainless steel screen; and, crushing the porcine cancellous bone mineral porous scaffolds treated in the step (2-3) by a food crushing machine, and screening particulate porcine cancellous bone mineral porous scaffolds in 0.2-0.8 mm and 0.33-1 mm for further use by a stainless steel screen.

In view of the problem that the existing substitute materials for bone transplantation are difficult to have good three-dimensional interconnected mesh structure, mechanical strength, degradability, osteogenic activity and the like, in the present invention, a modified biological bone mineral scaffold doped based on lithium magnesium phosphate is obtained by using bovine (porcine) cancellous bone mineral highly similar to the human cancellous bone mineralized cell matrix in structure and composition as the precursor and the main source of calcium and phosphorus, doping one or more of other osteogenic active ions of strontium, zinc, iron, calcium and the like on the basis of active ions magnesium, lithium and phosphorus, and using white granulated sugar as the adhesive and pore forming agent, in order to select more ideal scaffolds for bone tissue engineering and substitute materials for bone transplantation.

The modified biological bone mineral scaffold doped based on lithium magnesium phosphate obtained in the present invention can effectively stabilize the doped magnesium lithium phosphate and one or more of other osteogenic active ions of strontium, zinc, iron and calcium, and maintain the three-dimensional interconnected mesh structure and natural crystal structure, and relatively good mechanical strength of the calcined bovine or porcine cancellous bone mineral porous scaffold. Moreover, the honeycomb-like and Ganoderma lucidum-like calcium phosphate crystals containing active ions grow on the wall of the scaffold. Thus, the specific surface area of the material can be effectively increased, and the cell adhesion and osteogenesis can be improved. With regard to the modified biological bone mineral scaffold doped based on lithium magnesium phosphate provided by the present invention, among particles that form the modified biological bone mineral scaffold, particles in 0.25-1 mm, 1-2 mm, 2-4 mm and 4-6 mm have good micro-pore structures, and there is good porosity among particles. The small particles have good hydrophilicity with the particulate dosage form, can be partially dissolved, and can be used as good carriers for drugs such as antibiotics.

### BRIEF DESCRIPTION OF DRAWINGS

FIG. 1 is a general photograph of two types of modified scaffolds according to the present invention;

FIG. 2 is an XRD analysis chart of the material according to the present invention;

FIG. 3 is an SEM graph of the product according to an embodiment of the present invention;

FIG. 4 is an SEM graph of calcium phosphate crystals doped with osteogenic active ions according to an embodiment of the present invention;

FIG. 5 is an energy spectrum of a modified material according to the present invention;

FIG. 6 is a diagram of the bacterial culture of the product of the present invention as an antibacterial agent carrier;

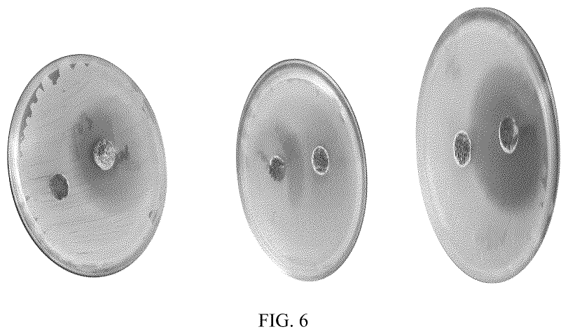

FIG. 7 is a diagram of the product of the present invention in combination with cell culture;

FIG. 8 is a diagram of the product of the present invention in a skull defect model experiment of an animal; and

FIG. 9 is a diagram showing the mechanical strength of the product of the present invention.

### DETAILED DESCRIPTION OF THE EMBODIMENTS

The preferred embodiments of the present invention will be illustrated below with reference to the drawings. It should be understood that the preferred embodiments described herein are merely used for illustrating and explaining the present invention, rather than limiting the present invention.

### Embodiment 1

This embodiment provides a modified biological bone mineral scaffold doped based on lithium magnesium phosphate, which is obtained by immersing a calcined bovine or porcine cancellous bone mineral porous scaffold into a solution containing one or more of active metal ions of magnesium, lithium, strontium, zinc, iron and calcium and a phosphorus source composite solution for hydrothermal reaction, baking, drying and calcining at a high temperature. An alternative for the immersing a calcined bovine or porcine cancellous bone mineral porous scaffold into a solution containing one or more of active metal ions of magnesium, lithium, strontium, zinc, iron and calcium and a phosphorus source composite solution for hydrothermal reaction is: immersing the calcined bovine or porcine cancellous bone mineral porous scaffold into a metal ion source solution containing one or more of active metal ions of magnesium, lithium, strontium, zinc, iron and calcium and a white granulated sugar solution, drying off the liquid by microwave or a constant temperature box, baking and drying at 96° C. to 198° C., and then putting into the phosphorus source composite solution for hydrothermal reaction. The phosphorus source composite solution is a phosphorus source binary system, so as to form a phosphorus source binary system containing osteogenic active metal ions. The hydrothermal reaction is in a constant temperature hydrothermal mode, controlled at 60° C. to 100° C. for 24 to 48 hours.

In the modified biological bone mineral scaffold doped based on lithium magnesium phosphate provided in this embodiment, the material-to-liquid ratio of the calcined bovine or porcine cancellous bone mineral porous scaffold to the metal ion source solution may be 15-50 g: 100 mL, and the material-to-liquid ratio of the calcined bovine or porcine cancellous bone mineral porous scaffold to the phosphorus source composite solution may be 15-50 g: 100 mL.

In the modified biological bone mineral scaffold doped based on lithium magnesium phosphate provided in this embodiment, in the metal ion source solution, the magnesium source is one of magnesium acetate, magnesium sulfate and magnesium hydrogen phosphate, etc.; the lithium source is lithium chloride; the calcium source is one of calcium chloride and calcium hydroxide; the zinc source is one of soluble zinc salts, such as zinc nitrate or zinc acetate; the strontium source is one of soluble strontium salts, such as strontium nitrate, strontium acetate or strontium sulfate; the iron source is one of soluble iron salts, such as ferrous sulfate, ferrous chloride, ferric trichloride or ferric acetate; the phosphorus source binary system is phosphoric acid and a soluble phosphate composite solution; and, the soluble phosphate composite solution is one or a combination of diammonium hydrogen phosphate, ammonium dihydrogen phosphate, disodium hydrogen phosphate, sodium dihydrogen phosphate, dipotassium hydrogen phosphate, potassium dihydrogen phosphate and magnesium hydrogen phosphate.

The final concentration of magnesium ions in the phosphorus source binary system containing osteogenic active metal ions is 0.05 to 0.20 mol/L; the final concentration of lithium ions in the phosphorus source binary system containing osteogenic active metal ions is 0.06 to 0.6 mol/L; the final concentration of zinc ions in the phosphorus source binary system containing osteogenic active metal ions is 0.1 to 0.6 mol/L; the final concentration of strontium ions in the phosphorus source binary system containing osteogenic active metal ions is 0.15 to 0.9 mol/L; the final concentration of ferrous ions in the phosphorus source binary system containing osteogenic active metal ions is 0.1 to 0.6 mol/L; the final concentration of supplementary calcium ions in the phosphorus source binary system containing osteogenic active metal ions is 0.15 to 1.5 mol/L; preferably, the final concentration of phosphate radials provided by phosphoric acid in the phosphorus source binary system containing osteogenic active metal ions is 0.15 to 0.9 mol/L; the final concentration of phosphate radials provided by soluble phosphate in the phosphorus source binary system containing osteogenic active metal ions is 0.06 to 0.6 mol/L; and, preferably, the ratio of the total molar concentration of osteogenic active cations including calcium in the reaction system (the molar number of monovalent lithium ions is halved, and the molar concentration of trivalent iron ions is multiplied by 1.5) to the molar concentration of phosphorus ions is 1.1-1.6:1.

After hydrothermal reaction, the liquid is dried off at a constant temperature, and baking and drying are performed at 75° C. to 198° C.

During the high-temperature calcination, the high-temperature calcination is performed at 750° C. to 1200° C. for 6 to 24 hours.

In the modified biological bone mineral scaffold doped based on lithium magnesium phosphate provided in this embodiment, a method for preparing the calcined bovine or porcine cancellous bone mineral porous scaffold is as follows:
(1): strip or block-shaped calcined bovine or porcine cancellous bone mineral porous scaffold
(1-1): cutting a bovine or porcine cancellous bone into cancellous bone strips or blocks with a thickness of 0.5 to 5 cm to obtain a raw material bone, where it is also possible to prepare a cylindrical calcellous bone strip by trephination;
(1-2): stewing the raw material bone for 36 to 60 min in distilled water in a pressure cooker, then cleaning with drinking water at 50° C. to 75° C., and repeating this step for 5 to 6 times; and
(1-3): drying the raw material bone treated in the step (1-2) for 12 to 24 hours in a constant temperature box at 80° C. to 120° C., then calcining the raw material bone for 6 to 12 hours in a calcination furnace at 900° C. to 1200° C., and then slowly cooling to the room temperature to obtain calcined cancellous bone mineral porous scaffolds, the powder diffraction components of the bovine or porcine cancellous bone mineral material being hydroxyapatite;
(2): particulate bovine or porcine cancellous bone mineral porous scaffold
(2-1): cutting bovine or porcine cancellous bone into bone strips or bone blocks with a thickness of 0.5 to 6 cm to obtain a raw material bone;
(2-2): stewing the raw material bone for 36 to 60 min in distilled water in a pressure cooker, then cleaning with drinking water at 50° C. to 75° C., and repeating this step for 5 to 6 times;
(2-3): drying the raw material bone treated in the step (2-2) for 12 to 24 hours in a constant temperature box at 80° C. to 120° C., then calcining the raw material bone for 6 to 12 hours in a calcination furnace at 900° C. to 1200° C., and then slowly cooling to the room temperature to obtain calcined bovine or porcine cancellous bone mineral porous scaffolds; and
(2-4): crushing the bovine cancellous bone mineral porous scaffolds treated in the step (2-3) by a food crushing machine, and screening granular bovine cancellous bone mineral porous scaffolds of various specifications such as 0.2-1 mm, 1-3 mm, 3-5 mm and 5-7 mm for further use by a stainless steel screen; and, crushing the porcine cancellous bone mineral porous scaffolds treated in the step (2-3) by a food crushing machine, and screening granular porcine cancellous bone mineral porous scaffolds in 0.2-0.8 mm and 0.33-1 mm for further use by a stainless steel screen.

Powder diffraction component analysis and inductively coupled plasma atomic emission spectrographic element analysis are performed on the bovine or porcine cancellous bone mineral.

The results of powder diffraction show that the components of bovine and porcine bone mineral is “elementary” hydroxyapatite (Ca5(PO4)3OH).

The results of inductively coupled plasma atomic emission spectrographic element detection are as follows:
Ca39.2; P18.1; Li0.016, Mg0.72; Sr0.058; Zn0.021; Fe0.03.

In this embodiment, during the specific implementation process, the specific process of immersing a calcined bovine or porcine cancellous bone mineral porous scaffold into a solution containing one or more of active metal ions of magnesium, lithium, strontium, zinc, iron and calcium and a phosphorus source composite solution for hydrothermal reaction, baking, drying and calcining at a high temperature to obtain samples will be further illustrated as below:

### Embodiment 2

[Sample added with lithium magnesium phosphate: No. 2104211]: 300 mL of composite solution containing 0.15 mol/L magnesium sulfate heptahydrate, 0.2 mol/L lithium chloride, 0.30 mol/L diammonium hydrogen phosphate and 0.30 mol/L orthophosphoric acid was prepared in a 500 mL flask and then completely dissolved for 3 minutes by microwave medium fire, and 50 g of the calcined bovine cancellous bone mineral porous scaffold with a porosity of 65% to 80% was immersed into the solution, where the total cation/phosphorus molar ratio in the reaction system was 1.3582. The reaction system was subjected to hydrothermal reaction for 24 hours at 100° C. under the protection of a 1000 mL flask, the protective flask was removed, and 10.76 g of 0.105 mol/L white granulated sugar was added in the solution. The reaction system was transferred to a 1000 mL flask, and then dried for 14 hours at 100° C. in a constant temperature box (stirred repetitively in the early stage). The temperature was increased by 2.5° C. every minute until the temperature was up to 900° C., and this temperature was maintained for 360 minutes and then reduced. The furnace temperature was reduced to 400° C. for 180 minutes, and then reduced to the room temperature for 90 minutes, so that the sample No. 2104211 was obtained. The sample was 61.52 g and had good texture and strength, and a small number of materials had non-volatilized carbon black.

The sample No. 2104211 was analyzed and detected:

Ca9MgLi(PO4)7 46.7%
Ca2(P2O7 52.0%
LiMgPO4 1.3%

The results of inductively coupled plasma atomic emission spectrographic element detection were as follows (wt %):
Li 0.53, Mg 1.64, Ca 33.6, P 22.4;
the molar ratio of lithium to total cation was 7.77%; and
the molar ratio of magnesium to total cation was 6.87%.

The results of powder diffraction component analysis and inductively coupled plasma atomic emission spectrographic element analysis indicated that lithium, magnesium and phosphorus were effectively doped.

### Embodiment 3

[Sampled added with lithium calcium magnesium phosphate: No. 2105262]: 200 mL of composite solution containing 0.1 mol/L anhydrous calcium chloride, 0.05 mol/L magnesium sulfate heptahydrate, 0.1388 mol/L lithium chloride and 0.04 mol/L white granulated sugar was prepared, and 50 g of the bovine cancellous bone mineral scaffold with a porosity of 60% to 80% was added. The liquid was dried off by microwave medium-low fire (stirred repetitively), and the scaffold was transferred to a 1000 mL flask and baked for 300 minutes at 198° C. in a constant temperature box (stirred repetitively in the early stage) until the scaffold became black brown and was completely dried for further use. 200 mL of composite solution containing 0.225 mol/L orthophosphoric acid (3 mL of orthophosphoric acid) and 0.0909267 mol/L diammonium hydrogen phosphate was prepared, and the calcined bovine cancellous bone mineral porous scaffold treated with lithium calcium magnesium and white granulated sugar was immersed in the solution [the molar ratio of total osteogenic active metal ions to phosphorus in the reaction system was 1.4975], then subjected to hydrothermal reaction for 24 hours at 85° C. [under the protection of a 1000 mL flask], dried at 198° C. [stirred repetitively] and baked for 5 hours at 198° C. [stirred repetitively in the early stage]. After the surface of the material was carbonized [58.37 g], the temperature was increased by 2.5° C. every minute up to 900° C., and this temperature was maintained for 360 minutes, then reduced to 400° C. for 180 minutes, and reduced to the room temperature 70° C. for 90 minutes, so that the sample doped with lithium calcium magnesium was obtained: No. 2105262 [53.76 g, with good appearance, texture, strength, air permeability and light transmittance].
Lithium Calcium Magnesium Phosphate Ca9MgLi(PO4)7
Hydroxylapatite, syn Ca5(PO4)3OH
Calcium diphosphate-β|Calcium Phosphate Ca2(P2O7)

The sample No. 2105262 was detected:

Ca9MgLi(PO4)7 59.7%
Ca5(PO4)3OH 28.9%
Ca2(P2O7) 11.4%

The result of powder diffraction component analysis indicated that lithium, magnesium, calcium and phosphorus were effectively doped.

### Embodiment 4

[Sampled added with lithium calcium magnesium phosphate: No. 2105202]: 200 mL of composite solution containing 0.1 mol/L anhydrous calcium chloride, 0.05 mol/L magnesium sulfate heptahydrate, 0.06 mol/L lithium chloride and 0.04 mol/L white granulated sugar was prepared, and 50 g of the bovine cancellous bone mineral particulate scaffold with a diameter of 2 to 4 mm was immersed into the solution. The liquid was dried off for repetitive two minutes by microwave medium-low fire (stirred repetitively), and the scaffold was transferred to a 1000 mL flask and baked for 300 minutes at 198° C. in a constant temperature box (stirred repetitively) until the scaffold became black brown and was completely dried for further use. 200 mL of composite solution containing 0.225 mol/L orthophosphoric acid (3 mL of orthophosphoric acid) and 0.06466 mol/L diammonium hydrogen phosphate was prepared, and the calcined bovine cancellous bone mineral porous scaffold treated with lithium calcium magnesium was immersed in the solution [the molar ratio of total divalent ions to phosphorus ions in the reaction system was 1.49749], then subjected to hydrothermal reaction for 24 hours at 85° C. [under the protection of a 1000 mL flask], dried at 198° C. without protection [stirred repetitively] and baked for 2 hours at 198° C. After the surface of the material was carbonized, the temperature was increased by 2.5° C. every minute up to 1075° C., and this temperature was maintained for 360 minutes, then reduced to 400° C. for 400 minutes, and reduced to the room temperature 70° C., so that the sample doped with lithium calcium magnesium was obtained: No. 2105202 [52.79 g, with good appearance, texture and strength].

The results of powder diffraction of the sample No. 2105202 were as follows:

Ca9MgLi(PO4)7 46.9%
Ca5(PO4)3OH 53.1%

The results of inductively coupled plasma atomic emission spectrographic element detection of the sample No. 2105202 were as follows (wt %):
Li 0.19, Mg1.42, Ca 35.8, P 20.2.
the molar ratio of magnesium to total cation≈5.96%
the molar ratio of lithium to total cation≈2.84%

The results of powder diffraction component analysis and inductively coupled plasma atomic emission spectrographic element analysis indicated that lithium, magnesium and phosphorus were effectively doped.

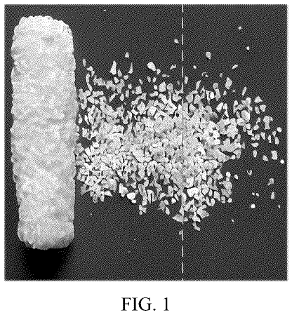

### Embodiment 5

[Sample added with lithium magnesium strontium phosphate: No. 2104191]: 300 mL of composite solution containing 0.05 mol/L magnesium sulfate heptahydrate, 0.10 mol/L strontium nitrate [6.3489 g of strontium nitrate], 0.25 mol/L lithium chloride [3.17925 g], 0.15 mol/L diammonium hydrogen phosphate [5.9427 g of diammonium hydrogen phosphate], 0.3 mol/L orthophosphoric acid [6 mL of orthophosphoric acid] was prepared (the molar ratio of total metal ions/phosphorus ions in the reaction system was 1.3391), and 50 g of the calcined bovine cancellous bone mineral porous scaffold with a porosity of 60% to 80% was immersed into the solution. The solution scale was 325 ml. The hydrothermal reaction was performed under protection for 12 hours and then performed without protection, and 0.165 mol/L white granulated sugar [10.76 g] was added. The turbid solution at the bottom was intermittently sucked and then sprayed on the scaffold for 12 hours. The scaffold was dried for 12 hours at 100° C. until the surface of the material was carbonized [initially stirred for multiple times]. The temperature was increased by 2.5° C. every minute up to 900° C., and this temperature was maintained for 360 minutes, then reduced to 400° C. for 180 minutes, and reduced to the room temperature for 90 minutes, so that the sample doped with lithium magnesium strontium was obtained: No. 2104191 [58.97 g, with good appearance, texture and strength, and good micro-pore structure shown in FIG. 1].

The results of powder diffraction detection of the sample No. 2104191 were as follows:

Ca9MgLi(PO4)7 88.4%
Ca2(P2O7) 11.6%

The results of inductively coupled plasma atomic emission spectrographic element analysis of the sample No. 2104191 were as follows (wt %):
Li 0.77, Mg 1.57, Ca 32.6, P/Sr 4.08.
the molar ratio of lithium to total cation≈10.74%
the molar ratio of magnesium to total cation≈5.98%
the molar ratio of strontium to total cation≈5.98%

The results of powder diffraction component analysis and inductively coupled plasma atomic emission spectrographic element analysis indicated that lithium, magnesium, strontium and phosphorus were effectively doped.

### Embodiment 6

[Sample added with lithium magnesium strontium calcium: No. 2105261]: a composite solution containing 0.1 mol/L anhydrous calcium chloride, 0.05 mol/L magnesium sulfate heptahydrate, 0.1388 mol/L lithium chloride, 0.12 mol/L strontium nitrate and 0.04 mol/L white granulated sugar was prepared with distilled water, and 50 g of the bovine cancellous bone mineral scaffold with a porosity of about 75% to 90% was immersed into the solution. The liquid was dried off by microwave medium-low fire, and the scaffold was baked for 300 minutes at a constant temperature of 198° C. in the original 500 mL flask until the scaffold became black brown for further use.

200 mL of composite solution containing 0.225 mol/L orthophosphoric acid and 0.1709267 mol/L diammonium hydrogen phosphate was prepared, and the calcined bovine cancellous bone mineral porous scaffold treated with lithium magnesium strontium calcium and the like was immersed into the solution (the molar ratio of osteogenic active ions to phosphorus in the reaction system was 1.4961), then subjected to hydrothermal reaction at 85° C. [under the protection of a 1000 mL flask for 24 hours, and the solution was dried without protection at 198° C.], and braked for 5 hours at a constant temperature of 198° C. The surface of the material was carbonized [60.40 g]. The temperature was increased by 2.5° C. every minute up to 900° C., and this temperature was maintained for 360 minutes and then reduced to 400° C. for 180 minutes, and reduced to the room temperature 70° C. for 180 minutes, so that the sample added with lithium magnesium strontium calcium was obtained: No. 2105261 [56.31 g, with good appearance, texture and strength; a small number of materials had non-volatilized carbon black].

The results of powder diffraction detection of the sample No. 2105261 were as follows:

Ca9MgLi(PO4)7 61.7%
Ca5(PO4)3OH 30.1%
Ca2(P2O7) 8.2%

### Embodiment 7

[Sample added with lithium magnesium strontium calcium: No. 2106151]: 200 mL of composite solution containing 0.10 mol/L anhydrous calcium chloride, 0.055 mol/L magnesium sulfate heptahydrate, 0.576 mol/L lithium chloride trihydrate, 0.12 mol/L strontium nitrate and 0.06 mol/L white granulated sugar was prepared, and 50 g of the bovine cancellous bone mineral scaffold [calcined at 988° C.] was immersed into the solution. The liquid was drilled by microwave medium fire and thawing fire, and the scaffold was dried for 600 minutes in a constant temperature box at 198° C. in the original 500 mL flask [stirred repetitively] until the scaffold [62.14 g] became black for further use.

200 mL of composite solution containing 0.45 mol/L orthophosphoric acid (6 mL of orthophosphoric acid) and 0.17733 mol/L diammonium hydrogen phosphate was prepared, and the calcined bovine cancellous bone mineral porous scaffold treated with lithium magnesium strontium calcium was immersed in the solution [the molar ratio of calcium and other osteogenic active ions [by divalent ions] to phosphorus in the reaction system was 1.335418], then subjected to hydrothermal reaction for 24 hours at 70° C. [under the protection of a 1000 mL flask], dried by microwave medium fire and thawing fire [stirred repetitively] and baked for 6 hours at 198° C. [stirred repetitively]. After the surface of the material was carbonized [63.05 g], the temperature was increased by 2.5° C. every minute up to 900° C., and this temperature was maintained for 360 minutes, then reduced to 400° C. for 180 minutes, and reduced to the room temperature 70° C. for 180 minutes, so that the sample with lithium magnesium strontium calcium was obtained: No. 2106151 [57.26 g, with good appearance, texture and strength].

The sample No. 2106151 was subjected to powder diffraction and element analysis:

Ca9MgLi(PO4)7 78.4%
Ca5(PO4)3(OH) 11.2%
LiCa(PO4) 7.4%
Li3PO4 3.1%

The results of inductively coupled plasma atomic emission spectrographic element detection of the sample No. 2106151 were as follows (wt %):
Li 1.18; Mg 1.24; Ca 32.8; P 20.9; Sr 2.74.
the molar ratio of magnesium to total cation≈5.17%
the molar ratio of lithium to total cation≈17.2%
the molar ratio of strontium to total cation≈3.17%

The results of powder diffraction component analysis and inductively coupled plasma atomic emission spectrographic element analysis indicated that lithium, magnesium and phosphorus were effectively doped.

### Embodiment 8

[Sample added with lithium magnesium strontium calcium: No. 2106152]: 200 mL of composite solution containing 0.10 mol/L anhydrous calcium chloride, 0.055 mol/L magnesium sulfate heptahydrate, 0.5769 mol/L lithium chloride trihydrate, 0.12 mol/L strontium nitrate and 0.06 mol/L white granulated sugar was prepared, and 50 g of the bovine cancellous bone mineral scaffold was immersed into the solution. The liquid was dried off by microwave medium fire and thawing fire [stirred repetitively], and the scaffold was baked for 300 minutes in a constant temperature box at 198° C. in the original 500 mL flask until the scaffold became black brown [67.18 g] and was completely dried for further use.

200 mL of composite solution containing 0.45 mol/L orthophosphoric acid [6 mL of orthophosphoric acid] and 0.17733 mol/L diammonium hydrogen phosphate was prepared, and the bovine cancellous bone mineral porous scaffold treated with calcium magnesium lithium strontium and white granulated sugar was immersed into the solution [the ratio of the molar concentration of metal ions (by divalent ions) to the molar concentration of phosphorus ions in the reaction system was set as 1.35]. The scaffold was subjected to hydrothermal reaction for 24 hours at 70° C. [under the protection of a 1000 mL flask] and then dried for 6 hours [stirred repetitively] at 198° C. by microwave medium fire and thawing fire. The temperature of the scaffold with carbonized material surface [66.63 g] was increased by 2.5° C. every minute up to 900° C., and this temperature was maintained for 360 minutes and then reduced to 400° C. for 360 minutes, and reduced to the room temperature 70° C. for 360 minutes, so that the same with lithium magnesium strontium calcium was obtained: No. 2106152 [59.35 g, with good appearance, pore structure, and strength (higher than that of the precursor)].

The sample No. 2106152 was subjected to powder diffraction and inductively coupled plasma atomic emission spectrographic element detection:

Ca9MgLi(PO4)7 79.5%
Li3PO4 8.5%
Ca2(P2O7 12.0%)

The results of inductively coupled plasma atomic emission spectrographic element detection of the sample No. 2106152 were as follows (wt %):
Li 1.15, Mg 1.21, Ca 32.1, P 21.1, Sr 2.66.
the molar ratio of magnesium to total cation was 5.21%
the molar ratio of lithium to total cation was 16.57%
the molar ratio of strontium to total cation was 3.71%

The results of powder diffraction component analysis and inductively coupled plasma atomic emission spectrographic element analysis indicated that lithium, magnesium, strontium, calcium and phosphorus was effectively doped.

### Embodiment 9

[Sample added with lithium magnesium strontium calcium: No. 2106281]: 200 mL of composite solution containing 0.10 mol/L anhydrous calcium chloride, 0.055 mol/L magnesium sulfate heptahydrate, 0.36 mol/L lithium chloride trihydrate, 0.3 mol/L strontium nitrate and 0.06 mol/L white granulated sugar was prepared, and 50 g of the bovine cancellous bone mineral scaffold [calcined at 988° C.] was immersed into the solution. The liquid was dried off by microwave medium fire and thawing fire, and the scaffold was baked for 600 minutes in a constant temperature box at 198° C. in the original 500 mL flask until the scaffold completely became black [70.27=71.68-1.41 g powder] for further use.

200 mL of composite solution containing 0.45 mol/L orthophosphoric acid (6 mL of orthophosphoric acid) and 0.18836 mol/L diammonium hydrogen phosphate was prepared, and the calcined bovine cancellous bone mineral porous scaffold treated with calcium magnesium lithium strontium and white granulated sugar was immersed in the solution [the molar ratio of total metal ions to phosphorus in the reaction system was 1.389], then subjected to hydrothermal reaction for 24 hours at 60° C. [under the protection of a 1000 mL flask], dried by microwave medium fire and thawing fire and baked for 6 hours at 198° C. [stirred repetitively]. After the surface of the material was carbonized [69.35 g=73.02-3.67 g powder], the temperature was increased by 2.5° C. every minute up to 900° C., and this temperature was maintained for 360 minutes, then reduced to 400° C. for 180 minutes, and reduced to the room temperature 70° C. for 180 minutes, so that the sample doped with lithium magnesium strontium calcium phosphate was obtained: No. 2106281. The scaffold was 62.8 g.

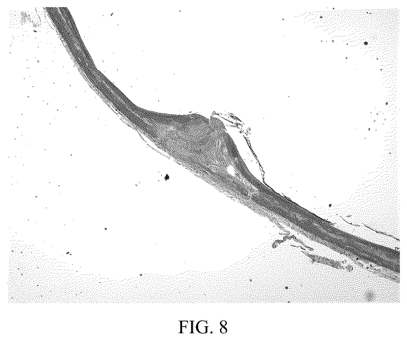

The product in this embodiment in combination with cell culture was shown in FIG. 8. By staining cells and scaffolds and analyzing by confocal microscopy, the biological activity and surface nano-lattice structure of the artificial bone scaffold could promote the adhesion and spreading of cells on the surface of artificial bone and promote the biological activity of cells.

The sample No. 2106281 was subjected to material powder diffraction and element detection:

Ca9MgLi(PO4)7 69.7%
Ca10.132(PO4)5.958 (OH)3.258 30.3%

The results of inductively coupled plasma atomic emission spectrographic element detection of the sample No. 2106281 were as follows (wt %):
Li0.64, Mg 1.04, Ca 29.1, P 18.6, Sr6.9.
the molar ratio of magnesium to cations was 4.7829%
the molar ratio of lithium to cations was 10.3%
the molar ratio of strontium to cations was 8.767%

The results of powder diffraction component analysis and inductively coupled plasma atomic emission spectrographic element analysis indicated that lithium, magnesium, strontium and phosphorus were effectively doped.

### Embodiment 10

[Sample added with lithium magnesium strontium calcium: No. 2106081]: 200 mL of composite solution containing 0.1 mol/L anhydrous calcium chloride, 0.05 mol/L magnesium sulfate heptahydrate, 0.1388 mol/L lithium chloride, 0.12 mol/L strontium nitrate and 0.05 mol/L white granulated sugar was prepared, and 50 g of the bovine cancellous bone mineral scaffold was immersed into the solution. The liquid was dried off by microwave medium fire [stirred repetitively] and dried for 120 minutes in a constant temperature box at 136° C., and the scaffold was baked for 300 minutes in the constant temperature box at 198° C. and completely dried until the scaffold became black brown powder for further use [57.31 g].

200 mL of composite solution containing 0.225 mol/L orthophosphoric acid (3 mL of orthophosphoric acid) and 0.098 mol/L diammonium hydrogen phosphate was prepared, and then poured into the flask with the calcined bovine cancellous bone mineral porous scaffold treated with calcium magnesium lithium and white granulated sugar [the molar ratio of total cation to phosphorus in the reaction system was 1.556]. The reaction system was subjected to hydrothermal reaction for 27 hours at 85° C. [under the protection of a 1000 mL flask], and the solution was dried for 5 hours at 108° C. without protection [the turbid solution at the bottom was intermittently sucked and then spayed on the scaffold]. The scaffold was then baked for 5 hours at 198° C. until the surface of the material is completely carbonized [58.10 g]. The temperature was increased by 2.5° C. every minute up to 800° C. for 385 minutes, and this temperature was maintained for 360 minutes and then reduced to 400° C. for 360 minutes, and reduced to the room temperature 35° C. for 360 minutes, so that the sample doped with lithium magnesium strontium calcium phosphate was obtained: No. 2106081:54.22 g, with good appearance, texture and strength. A small number of materials had non-volatilized carbon black.
Hydroxylapatite, syn Ca5(PO4)3(OH)
Whitlockite magnesian, syn Ca18Mg2H2(PO4)1

The sample No. 2106081 was subjected to material powder diffraction and element detection:

Ca5(PO4)3(OH) 75.3%
Ca18Mg2H2(PO4)14 24.7%

### Embodiment 11

[Sampled added with lithium magnesium calcium phosphate: No. 2111231] 100 mL of composite solution containing 1.8 mol/L anhydrous calcium chloride (19.98 g), 0.2 mol/L magnesium acetate tetrahydrate (24.29 g), 0.36 mol/L lithium chloride trihydrate (1.516 g), 0.45 mol/L strontium nitrate (9.52 g) and 0.06 mol/L white granulated sugar (4.1 g) was prepared, and 50 g of the bovine cancellous bone mineral scaffold was immersed into the solution. The liquid was dried off by microwave low fire [stirred repetitively], and the scaffold was baked for 300 minutes at a constant temperature of 176° C. in the original 1000 mL flask until the surface of the scaffold became black brown and mushy. The scaffold was baked for 360 minutes at 196° C. [powder and small particles were shed off] for further use.

If the reaction solution was set as 100 mL and the molar ration of calcium phosphate and active ions to phosphorus in the reaction system was set as 1.54676, the concentration of phosphate radicals in the reaction system except for the scaffold was 2.086666 mol/L, 6 mL of orthophosphoric acid and 15.66 mL of diammonium hydrogen phosphate was needed to prepare a composition solution containing 0.9 mol/L orthophosphoric acid and 1.18667 mol/L diammonium hydrogen phosphate, and the calcined bovine cancellous bone mineral porous scaffold treated with lithium magnesium calcium strontium was immersed into the solution. The reaction system was subjected to hydrothermal reaction at 75° C. [under the protection of a 2000 mL flask]. After half of an hour, white oval new substances were uniformly distributed around the scaffold. 24 hours later [the solution was dried after 23 hours, and no protection was needed in the last hour]. After a small part [divided into two parts 2111231 and 21112312] was taken out, the remaining part was dried for 2 hours at 126° C. without protection. After a small part was taken out [2111232], the remaining part was dried for 2 hours at 198° C. [2111233, 2111234]. The samples 2111231 and 2111232 were heated by 5° C. every minute up to 1075° C. [the room temperature was set as 20° C.], and this temperature was maintained for 6 hours and then reduced [the temperature was set to be reduced to 150° C. for 360 minutes] [good strength, possibly good elasticity modulus, low brittleness, good appearance and air permeability; the sample 2111231 was 9.19 g, and the sample 2111232 was 13.64 g]. The samples 2111231 and 2111233 were heated by 5° C. every minute up to 750° C. for 132 minutes, maintained for 720 minutes and then reduced to 100° C. for 360 minutes, and automatically reduced to the room temperature after 1 minute, to obtain samples doped with lithium magnesium strontium calcium phosphate: No. 2111231 (7.63 g) and No. 2111233 (19.02 g) [with good appearance, texture and strength; there were non-volatilized carbon black in a small number of materials, but the strength was higher than that of the sample calcined at 1075° C.].

The sample 2111234 was heated by 5° C. every minute up to 900° C. for 172 minutes, and this temperature was maintained for 480 minutes and then reduced to 100° C. for 360 minutes, and reduced to the room temperature 10° C. for 180 minutes, so that the sample 2111234 was obtained [22.05 g, with good appearance, texture and strength; there were non-volatilized carbon black in a small number of materials] [the total weight of the scaffold was 71.53 g, which is 1.4306 times of the weight (50 g) of the precursor]. The sample was subjected to powder diffraction and element analysis. After scanning electron microscopy and energy spectrum detection, in the sample 211123, the calcium phosphate crystals with good active ions looked like an ant nest, so that the specific surface area of the material could be further improved, and it was beneficial for the adhesion of bone repair cells. The results of powder diffraction of the sample 2111234 were as follows: Magnesium Phosphate (Ca2.589 Mg0.411)(PO4)2; Calcium diphosphate—β|Calcium Phosphate Ca2(P2O7) Dicalcium diphosphate (V)—α|Calcium Phosphate Ca2P207

2111231 [1075° C.]
(Ca2.589Mg0.411)(PO4)2 73.7%
Ca2(P2O7) 26.3%
2111232 [1075° C.]
(Ca2.589Mg0.411)(PO4)2 90.8%
Ca2(P2O7) 9.2%
2111233 [750° C.]
Ca9MgLi(PO4)7 23.1%
Ca2(P2O7) 27.5%
Ca5(PO4)3(OH) 49.5%
2111234 [900° C.]
(Ca2.589Mg0.411)(PO4) 34.7%
Ca2(P2O7) 21.9%
Ca2P2O7 43.4%

The results of inductively coupled plasma atomic emission spectrographic element detection were as follows (wt %):

2111234 Lithium 0.51; magnesium 0.99; strontium 4.41; calcium 33.0; phosphorus 21.7.
the molar ratio of magnesium to total cation≈4.166%
the molar ratio of lithium to total cation≈7.42%
the molar ratio of strontium to total cation≈5.0532%

The results of powder diffraction component analysis and inductively coupled plasma atomic emission spectrographic element analysis indicated that lithium, magnesium, strontium, calcium and phosphorus were effectively doped.

### Embodiment 12

[Sampled added with lithium magnesium strontium calcium phosphate: No. 2111291] 100 mL of composite solution containing 0.9 mol/L anhydrous calcium chloride, 0.2 mol/L magnesium acetate tetrahydrate, 0.36 mol/L lithium chloride trihydrate, 0.45 mol/L strontium nitrate and 0.06 mol/L white granulated sugar was prepared, and 50 g of the bovine cancellous bone mineral scaffold was immersed into the solution. The liquid was dried off by microwave low fire [stirred repetitively], and the scaffold was dried for 180 minutes at a constant temperature of 75° C. in the original 500 mL flask and baked for 300 minutes at 196° C. in a crucible [the scaffold became black brown, and sand-like particles were shed off] for further use.

100 mL of reaction solution was prepared in a new 500 mL flask (the molar ratio of calcium and active ions to phosphorus in the reaction system was set as 1.55). The concentration of phosphate radicals in the reaction system except for the scaffold should be 1.342 mol/L. A composite solution containing 0.6 mol/L orthophosphoric acid and 0.7342 mol/L diammonium hydrogen phosphate was prepared and then completely dissolved for 4 minutes by microwave medium fire, and the calcined bovine cancellous bone mineral porous scaffold treated with lithium magnesium calcium strontium was immersed into the solution. The reaction system was subjected to hydrothermal reaction for 24 hours at 75° C. [under the protection of a 2000 mL flask; after half an hour, it was seen that white oval new substances were uniformly distributed around the scaffold]. 24 hours later, the liquid was dried off at 75° C., and the reaction system was baked at 196° C. in a crucible [the scaffold was evenly black brown and 84.42 g, and was evenly divided into two parts, i.e., 2111291 and 2111292].

The sample 2111291 was heated by 5° C. every minute up to 900° C. for 172 minutes, and this temperature was maintained for 720 minutes, then reduced to 100° C. for 360 minutes and reduced to the room temperature after 1 minute [32.53/42.21/25 g, good appearance, texture and strength; there were new particles generated on the wall of the scaffold under a magnifying lens]. The sample 2111292 was heated by 5° C. every minute up to 750° C. for 148 minutes [the furnace temperature was 72° C.], maintained for 720 minutes, then reduced to 100° C. for 360 minutes and reduced to the room temperature after 1 minute, to obtain the sample 2111292 [good appearance, texture and strength; a small number of materials had non-volatilized carbon black].

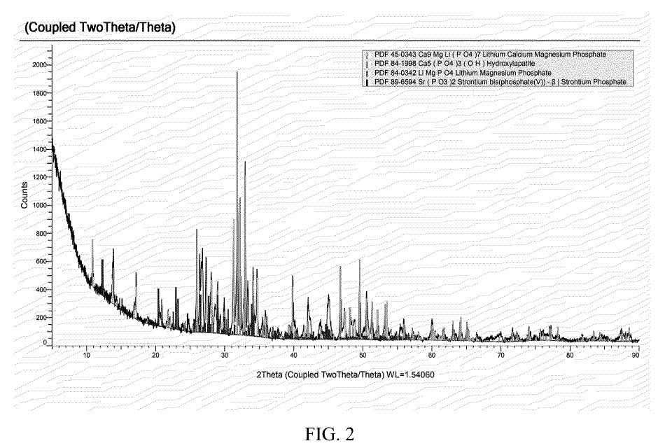

The results of powder and element analysis of the samples 2111291 and 2111292 indicated that the elements such as lithium, magnesium, strontium and phosphorus were effectively doped, as shown in FIG. 2.

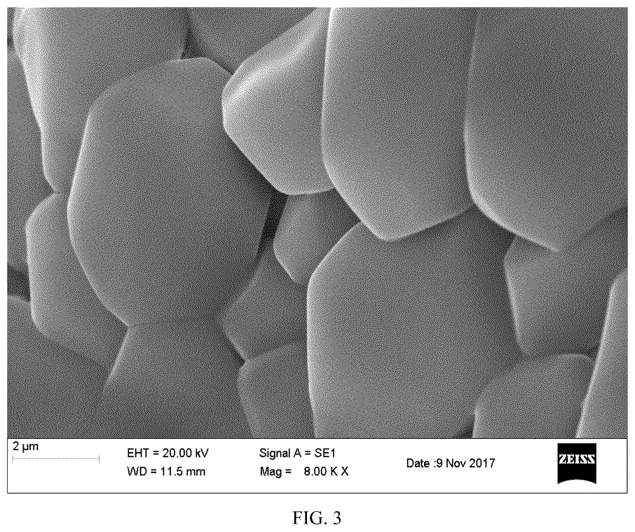

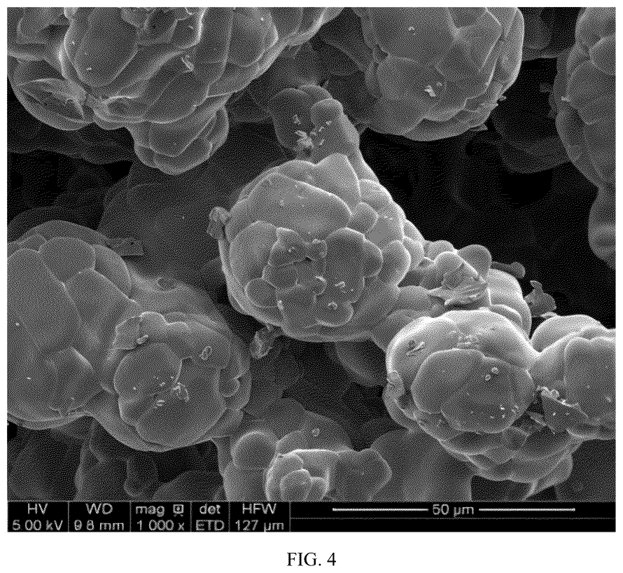

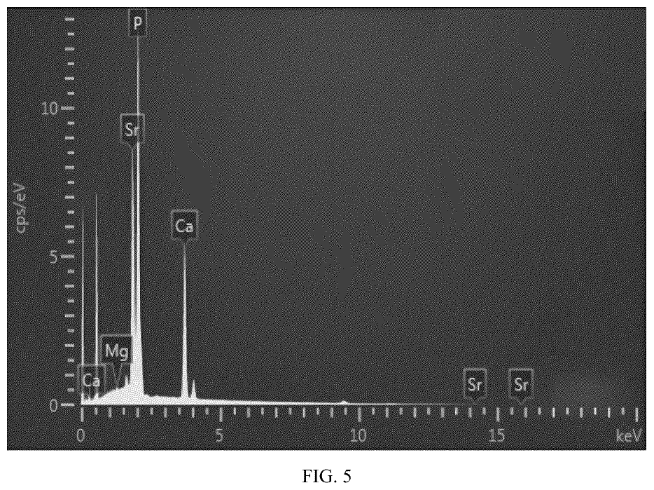

The sample No. 2111291 was subjected to scanning electron microscopy and energy spectrum detection, showing that calcium phosphate crystals with active ions grew uniformly and the calcium phosphate crystals were Ganoderma lucidum-like. Thus, the specific surface area of the material could be further improved, and it was beneficial or the adhesion and spreading of bone repair cells for scanning electron microscopy, as shown in FIGS. 3, 4 and 5.

2111291 [900° C.]
(Ca2.589Mg0.411)(PO4)2 55.7%
Ca5(PO4)3OH 38.0%
Sr(PO3)2 6.3%
2111292 [750° C.]
Ca9MgLi(PO4)7 27.1%
Ca5(PO4)3(OH) 53.2%
Li MgPO47.1%
Sr(PO3)2 12.6%

The results of inductively coupled plasma atomic emission spectrographic element detection were as follows (wt %):
2111291 Lithium 0.55; magnesium 1.02; strontium 4.64; calcium 34.5; phosphorus 20.4.

The results of powder diffraction component analysis and inductively coupled plasma atomic emission spectrographic element analysis indicated that lithium, magnesium, strontium, calcium and phosphorus were effectively doped.

### Embodiment 13

[Sampled added with lithium magnesium strontium: No. 2112121] 100 mL of composite solution containing 0.225 mol/L, 0.1 mol/L magnesium acetate tetrahydrate, 0.18 mol/L lithium chloride trihydrate, 0.225 mol/L strontium nitrate (4.762) and 0.06 mol/L white granulated sugar was prepared with distilled water, and 25 g of the porcine cancellous bone mineral particulate [φ0.2-0.8 mm] scaffold was immersed into the solution. The liquid was dried off by microwave low fire [stirred repetitively], and the scaffold was dried for 120 minutes at a constant temperature of 75° C. in the original 500 mL flask and baked for 120 minutes at 196° C. in a crucible [the scaffold became black brown] for further use.

If the molar ratio of calcium and active ions to phosphorus ions in the reaction system was set as 1.55, the concentration of phosphate radicals in the reaction system except for the scaffold was 0.5258. 100 mL of composite solution containing 0.3 mol/L orthophosphoric acid and 0.2258 mol/L diammonium hydrogen phosphate was prepared in a new 500 mL flask, the calcined porcine cancellous bone mineral particles treated with lithium magnetism calcium strontium and white granulated sugar was poured into the new flask, and the scaffold was immersed with the composite solution (the molar ratio of calcium phosphate and active ions to phosphorus in the reaction system was about 1.35). The reaction system was subjected to hydrothermal reaction for 24 hours at 75° C. [under the protection of a 1000 mL flask] [maintained for 24 hours at 37° C.], the liquid was dried off at 75° C., and the scaffold was baked at 196° C. in a crucible [stirred intermittently; the scaffold became black brown finally and was 38.46 g. It was divided into two parts, i.e., 2112121 and 2112122, each of which was 19.23 g.

The sample 2112121 was heated by 3° C. every minute up to 750° C., maintained for 180 minutes, then reduced to 10° C. for 480 minutes, and reduced to the room temperature after 1 minute [16.56 g, good appearance, texture and strength; the surface seemed to be coated]. The sample 2112122 was heated by 5° C. every minute up to 1200° C. [heating for 126 minutes], and was maintained for 3 hours [it is set to cool to 15° C. for 636 minutes] so as to obtain the sample 2112122 [15.86 g, with good strength, possibly good elasticity modulus, low brittleness, and good appearance and air permeability].

After detection, the following was obtained:

2112121 [750° C.]
Ca9MgLi(PO4)7 45.7%
Ca5(PO4)3(OH) 54.3%
2112122 [1200° C.]
Ca2.86Mg0.14(PO4)2 100%

### Embodiment 14

[Sampled added with lithium magnesium strontium calcium phosphate: No. 2112151] 100 mL of composite solution containing 0.35 mol/L anhydrous calcium chloride, 0.1 mol/L magnesium acetate tetrahydrate, 0.18 mol/L lithium chloride trihydrate, 0.225 mol/L strontium sulfate and 0.06 mol/L white granulated sugar was prepared, and 25 g of the porcine cancellous bone mineral particulate [φ0.3-0.8 mm, composed of hydroxyapatite] scaffold was immersed into the solution. The liquid was dried off by microwave low fire [stirred repetitively], and the scaffold was dried for 120 minutes at a constant temperature of 75° C. in the original 500 mL flask and baked for 120 minutes at 196° C. in a crucible [the scaffold became black brown, and sand-like particles were shed off] for further use.

If the molar ratio of calcium and active ions to phosphorus ions in the reaction system was set as 1.5, the concentration of phosphate radicals in the reaction system except for the scaffold was 0.6766666. 100 mL of composite solution containing 0.3 mol/L orthophosphoric acid and 0.37666 mol/L diammonium hydrogen phosphate was prepared in a new 500 mL flask, and the calcined porcine cancellous bone mineral particles treated with lithium magnetism calcium strontium and white granulated sugar was immersed into the solution. The reaction system was subjected to hydrothermal reaction for 42 hours at 75° C., the liquid was dried off at 75° C., and the scaffold was baked at 196° C. in a crucible [stirred intermittently; the scaffold became black brown finally and was 37.05 g. It was divided into two parts, i.e., 2112151 [17.52] and 2112152 [17.53].

The sample 2112151 was heated by 3° C. every minute up to 900° C., maintained for 180 minutes, then reduced to 10° C. for 480 minutes, and reduced to the room temperature after 1 minute to obtain the sample 2112151 [15.44 g, good appearance, texture and strength; the surface seemed to be coated].

The sample 2112152 was heated by 5° C. every minute up to 1200° C. [heating for 396 minutes], and was maintained for 3 hours [it is set to cool to 15° C. for 536 minutes] so as to obtain the sample 2112152 [good strength, possibly good elasticity modulus, low brittleness, and good appearance and air permeability, 17.43 g].

The results of powder element detection and inductively coupled plasma atomic emission spectrographic element detection indicated that the material was effectively doped with elements.

2112151 [900° C.]
Ca9MgLi(PO4)7 52.4%
Ca5(PO4)3OH 47.6%
2112152 [1200° C.]
Ca5(PO4)3OH 17.4%
Ca2.86Mg0.14 (PO4)2 82.6%

The results of inductively coupled plasma atomic emission spectrographic element detection of the sample No. 2112152 were as follows (wt %):
Lithium 0.73, magnesium 1.24, strontium 5.58, calcium 33.2, phosphorus 20.7.

The results of powder diffraction component analysis and inductively coupled plasma atomic emission spectrographic element analysis indicated that lithium, magnesium, strontium, calcium and phosphorus were effectively doped.

### Embodiment 15

[Sample added with lithium zinc calcium phosphate: No. 2109061] 200 mL of composite solution containing 0.10 mol/L anhydrous calcium chloride, 0.08 mol/L magnesium acetate tetrahydrate, 0.18 mol/L lithium chloride and 0.18 mol/L zinc acetate dihydrate 219.5 was prepared, and 50 g of bovine cancellous bone mineral particles [0.4-1 mm, composed of hydroxyapatite, calcined at 988° C.] were immersed into the solution. The liquid was dried off by microwave medium fire and thawing fire (4.02 g of white granulated sugar was added at the appropriate time). The system was dried for 180 minutes in a constant temperature box at 176° C. in the original 500 mL flask and stirred into powder repetitively. The powder gradually became brown sand-like powder from caramel color for further use.

The molar ratio of total cation to phosphorus ions in the reaction system was set as 1.48. 200 mL of composite solution containing 0.3 mol/L orthophosphoric acid and 0.21836 mol/L sodium phosphate dibasic dodecahydrate was prepared, and the black sand-like bone mineral particles added with calcium magnesium strontium and white granulated sugar were immersed into the solution. The reaction system was subjected to hydrothermal reaction for 24 hours at 65° C., and the liquid was dried off at a low temperature by a microwave oven. The particles were baked for 6 hours at 176° C. and stirred every half hour, and the particles gradually became brown from caramel color for further use [65.64 g]. The temperature was increased by 5.8° C. every minute up to 900° C. [the room temperature was set as 30° C., and the temperature was increased for 150 minutes], and this temperature was maintained for 180 minutes, and reduced to 188° C. for 3 hours [30.77 g]. The volume was measured as 30 mL by a measuring cup, and the relative density of tricalcium phosphate was about 3.2 g/cm³, so that the porosity was calculated as about 67%. The particles were photographed by magnification and observed on the computer, and it was found that the modified biological scaffold particles with a diameter of 0.4-1 mm had micro-pores, as shown in FIG. 1.

The results of powder diffraction component analysis and inductively coupled plasma atomic emission spectrographic element analysis indicated that the elements such as lithium, magnesium, zinc and phosphorus of the material were effectively doped.

2109061
Ca9MgLi(PO4)7 72.7%
NaCaPO4 12.1%
Ca8.8(PO4)6(OH)1.92 15.2%

The results of inductively coupled plasma atomic emission spectrographic element detection were as follows (wt %):
Li 0.47, Mg 1.14, Ca 28.29, P 20.5, Zn2.39.

The results of powder diffraction component analysis and inductively coupled plasma atomic emission spectrographic element analysis indicated that lithium, magnesium, zinc, calcium and phosphorus were effectively doped.

### Embodiment 16

[Sample added with lithium magnesium zinc: No. 2108121] 200 mL of composite solution containing 0.6 mol/L zinc acetate dihydrate, 0.12 mol/L lithium chloride trihydrate, 0.1 mol/L magnesium acetate tetrahydrate and 0.06 mol/L white granulated sugar was prepared. 60 g of the scaffold prepared at 1075° C. was added, the solution was completely dried by medium fire in the microwave oven, and the scaffold was dried for 300 minutes in a constant temperature box at 196° C. in the original 500 mL flask [stirred more in the early stage] until the surface of the scaffold was uniformly carbonized for further use.

200 mL of composite solution containing 0.45 mol/L phosphoric acid and 0.6 mol/L diammonium hydrogen phosphate was prepared from 6 mL of phosphoric acid and 15.8472 g of diammonium hydrogen phosphate, then completely dissolved and then poured into a flask with the bone mineral scaffold added with zinc, lithium, magnesium and white granulated sugar [the solid-to-liquid ratio was 30 g: 100 mL, and the molar ratio of total cation to phosphorus ions in the reaction system was 1.298]. The reaction system was subjected to hydrothermal reaction for 24 hours at 65° C. [under the protection of a 1000 mL flask], the liquid was dried off by microwave after the hydrothermal reaction, and the reaction system was baked for 5 hours at 196° C. [stirred repetitively]. Then, the temperature was increased by 5° C. every minute up to 1075° C. [the room temperature was set as 25° C., and the temperature was increased for 210 minutes], and this temperature was maintained for 6 hours and then reduced [it is set to cool to 300° C. for 400 minutes] [good strength, good elasticity and appearance, low air permeability; the scaffold was 77.40 g, and the nodes added on the surface could be seen by the magnifying lens].

The results of powder diffraction component analysis and inductively coupled plasma atomic emission spectrographic element analysis indicated that the elements such as lithium, magnesium, zinc and phosphorus of the material were effectively doped.

2108121
Ca9MgLi(PO4)7 40.2%
Ca2.86Mg0.14(PO4)2 41.8%
Zn2P207 17.1%

The results of inductively coupled plasma atomic emission spectrographic element detection of the sample No. 2108121 were as follows (wt %):
Mg0.88; Ca26.7; P 19.6; Zn9.6.

The results of powder diffraction component analysis and inductively coupled plasma atomic emission spectrographic element analysis indicated that lithium, magnesium, zinc and phosphorus were effectively doped.

### Embodiment 17

[Sample added with lithium magnetism zinc calcium: No. 2107191] 200 mL of solution was prepared from 0.10 mol/L anhydrous calcium chloride, 0.06 mol/L magnesium acetate tetrahydrate, 0.24 mol/L lithium chloride trihydrate, 0.1 mol/L zinc acetate and 0.06 mol/L white granulated sugar was prepared, and 50 g of bovine cancellous bone mineral particles [0.4-1 mm, calcined at 988° C.] were added. The liquid was drilled by microwave medium fire and thawing fire, and the particles were dried for 300 minutes in a constant temperature box at 198° C. in the original 500 mL flask and stirred repetitively until the powder surface gradually became black for further use.

200 mL of composite solution containing 0.3 mol/L orthophosphoric acid and 0.165 mol/L diammonium hydrogen phosphate was prepared, and the black sand-like bone mineral particles added with calcium magnesium lithium zinc and white granulated sugar were immersed into the solution [the molar ratio of total cation to phosphorus ions in the reaction system was 1.38]. The reaction system was subjected to hydrothermal reaction for 27 hours and 28 minutes at 52° C., then dried at 2° C. and repetitively stirred to dry off the liquid. The scaffold was baked for 6 hours at 196° C., and was 64.45 g. The temperature was increased 2.5° C. by every minute up to 1075° C. [the room temperature was set as 25° C., and the temperature was increased for 420 minutes], and this temperature was maintained for 6 hours and then reduced [57.28 g, the particles were white and uniform, and there were micro-pores in the particles under the magnifying lens].

The sample 2107191 was detected to obtain the following:

Ca9MgLi(PO4)7 78.3%
Ca5(PO4)3OH 21.7%

### Embodiment 18

[Sample added with lithium magnesium zinc calcium phosphate: No. 2109071] 200 mL of 0.1 mol/L anhydrous calcium chloride, 0.08 mol/L magnesium acetate tetrahydrate, 0.09 mol/L lithium chloride and 0.18 mol/L zinc acetate was prepared, and 50 g of porcine cancellous bone mineral particles [0.33-1 mm, hydroxyapatite, calcined at 988° C.] were immersed into the solution. The liquid was dried off by microwave medium fire and thawing fire (4.1 g of white granulated sugar was added at the appropriate time). The reaction system was dried for 180 minutes in a constant temperature box at 176° C. in the original 500 Ml flask, and the powder was stirred repetitively until the powder gradually became brown sand-like powder from caramel color and was completely dried for further use.

200 mL of composite solution containing 0.3 mol/L orthophosphoric acid and 0.21836 mol/L disodium hydrogen phosphate was prepared, and then added into the flask with the brown sand-like bone mineral particles added with calcium magnesium sodium strontium calcium [the molar ratio of total cation to phosphorus in the reaction system was 1.4642]. The reaction system was subjected to hydrothermal reaction for 24 hours at 65° C. The liquid was dried off at a low temperature in the microwave oven, and the powder was baked for 3 hours at 176° C., and stirred every half hour until the powder gradually became brown sand-like powder from caramel color for further use.

The material was evenly divided into two parts, i.e., 2109071 and 21090711, each of which was 36.92 g. Wherein, the sample 2109071 was heated by 5.8° C. every minute up to 900° C. [heated for 150 minutes], maintained for 180 minutes, and cooled to 188° C. for 3 hours [33.99 g] [the material was sintered into a block which is difficult to disperse and has the same shape as the flask].

The sample 21090711 was heated by 5° C. every minute up to 1075° C. [the room temperature was set as 25° C., heated for 210 minutes], maintained for 180 minutes, and cooled to 188° C. for 220 minutes [33.99 g, good color] [the material was sintered into a block which is difficult to disperse and has the same shape as the flask, which might be well used in the block scaffold].

2109071 [900° C.]
Ca9MgNa(PO4)7 97.6%
CaH2P207 2.40%
21090711 [1075° C.]
Ca9MgLi(PO4)7 97.4%
NaCaPO4 2.60%

The results of inductively coupled plasma atomic emission spectrographic element analysis of the sample No. 2109071 were as follows (wt %):
Li 0.26; Mg 1.78; Ca 29.6; P 19.45; Zn4.71.

The results of powder diffraction component analysis and inductively coupled plasma atomic emission spectrographic element analysis indicated that lithium, magnesium, zinc, calcium and phosphorus were effectively doped.

### Embodiment 19

[Sample added with lithium magnesium iron phosphate: No. 2108291] 200 mL of composite solution containing 0.0675 mol/L magnesium acetate tetrahydrate, 0.12 mol/L lithium chloride trihydrate, 0.30 mol/L anhydrous ferric chloride and 0.06 mol/L white granulated sugar was prepared, and 50 g of the bovine cancellous bone mineral scaffold was added. The liquid was drilled by microwave medium fire and thawing fire, the reaction system was dried for 300 minutes in a constant temperature box at 198° C. in the original 500 mL flask, and the power was stirred repetitively until the powder gradually became black sands from caramel color for further use.

The ratio of total metal ions to phosphorus was set as 1.1:1. 200 mL of composite solution containing 6 mL of 0.45 mol/L orthophosphoric acid and 0.71136 mol/L ammonium hydrogen phosphate was prepared, and the black brown bone mineral scaffold treated with calcium magnesium lithium iron and white granulated sugar was added [under the protection of a 1000 mL flask], subjected to hydrothermal reaction for 24 hours at 70° C., dried by microwave thawing fire and baked for 3 hours at 196° C. [stirred repetitively]. The temperature was increased by 5° C. every minute up to 900° C. for 175 minutes, maintained for 360 minutes and reduced to 188° C. for 420 minutes, so that the sample 2108291 was obtained [73.44 g, uniform light green color, higher strength and low air permeability]. The sample was subjected to powder diffraction. The sample 2108291 was calcined for 8 hours at 1175° C. to obtain the sample 21082911 with beautiful color. By scanning electron microscopy and energy spectrum detection, it was found that the sample 21082911 had good calcium phosphate crystals containing active ions formed on the wall of the scaffold, and the new generated material was honeycomb-shaped.
LiFe(P2O7) Lithium iron (III) diphosphate|Lithium Iron Phosphate

2108291
Ca9MgLi(PO4)7 15.7%
LiFe(P207) 14.3%
Ca2(P207) 56.8%
Na2Mg(SO4)2(H2O)4 1.5%
Ca10.042(PO4)5.952(OH)2.292 11.7%
21082911
Ca9MgLi(PO4)7 65.2%
Ca2(P207) 44.61%

### Embodiment 20

[Sample added with lithium magnesium strontium iron phosphate: No. 2111161] 200 mL of composite solution containing 0.08 mol/L magnesium acetate tetrahydrate, 0.18 mol/L lithium chloride trihydrate, 0.15 mol/L anhydrous ferric chloride, 0.15 mol/L strontium nitrate and 0.06 mol/L white granulated sugar was prepared, and 50 g of the bovine cancellous bone mineral scaffold was immersed into the solution. The liquid was dried off by microwave thawing fire [the scaffold was orange], and the scaffold was baked and carbonized for 5 hours at 176° C. [63.4 g].

The molar ratio of divalent metal ions to phosphorus in the reaction system was set as 1.5 [the molar concentration of lithium chloride=2, the molar concentration of ferric trichloride×1.5]. If 200 ml of composite solution was prepared from 4 mL of phosphoric acid and 5.5 g of 0.20833 mol/L ammonium hydrogen phosphate, and the brown bone mineral scaffold treated with lithium magnesium strontium iron and white granulated sugar was immersed into the solution [under the protection of a 1000 mL flask], then subjected to hydrothermal reaction for 24 hours at 75° C., dried by microwave thawing fire and baked for 5 hours at 176° C. [stirred repetitively].

The temperature was increased by 5 every minute up to 1075° C., and this temperature was maintained for 6 hours and then reduced [it is set to cool to 75° C. for 5 hours] [good strength, possibly good elasticity modulus, low brittleness, and good appearance and air permeability].

The sample No. 2111161 was detected to obtain the following:

(Ca2.589Mg0.411)(PO4)2 97.42%
Ca9.868(PO4)5.586(OH)4.006 3.69%

The results of inductively coupled plasma atomic emission spectrographic element detection were as follows (wt %):
Lithium 0.37, magnesium 1.12, calcium 31.5, phosphorus 19.5, strontium 3.88, iron 1.53.
the molar ratio of lithium to total cation≈5.72%
the molar ratio of magnesium to total cation≈4.943%
the molar ratio of strontium to total cation≈4.784%
the molar ratio of iron to total cation≈2.94%

The results of powder diffraction component analysis and inductively coupled plasma atomic emission spectrographic element analysis indicated that lithium, magnesium, strontium, iron and phosphorus were effectively doped.

### Embodiment 21

[Sample added with lithium magnetism zinc iron calcium phosphate: No. 2107192] 200 mL of solution containing 0.10 mol/L anhydrous calcium chloride, 0.06 mol/L magnesium acetate tetrahydrate, 0.18 mol/L lithium chloride tetrahydrate, 0.09 mol/L zinc acetate, 0.09 mol/L ferric chloride and 0.06 mol/L white granulated sugar was prepared, and 50 g of porcine cancellous bone mineral particles [0.33-1 mm, hydroxyapatite, calcined at 988° C.] were added. The liquid was drilled by microwave medium fire and thawing fire, the reaction system was dried for 300 minutes in a constant temperature box at 198° C. in the original 500 mL flask, and the powder was stirred repetitively until the powder gradually became black sands from caramel color for further use.

200 mL of composite solution was prepared from 0.35 mol/L orthophosphoric acid and 0.21836 mol/L diammonium hydrogen phosphate, and the black sand-like bone mineral particles treated with calcium magnesium lithium strontium calcium and white granulate sugar was immersed into the solution [the molar ratio of total cation (the concentration of monovalent lithium ions=2, the concentration of trivalent iron ions×1.5)/phosphorus is 1.4219] and subjected to hydrothermal reaction for 25 hours and 28 minutes at 52° C. without a larger flask for protection. At the end of reaction, the solution just reached the plane of the scaffold. The particles were baked for 6 hours at 196° C. [stirred repetitively in the early stage] and became black brown [65.36 g], then heated by 2.5° C. every minute up to 1075° C. [the room temperature was set as 25° C., and the temperature was increased for 420 minutes], maintained for 6 hours and then cooled [the particles were white and 58.15 g, and the particles had micro-pores under the magnifying lens].

The sample No. 2107192 was detected to obtain the following:

Ca9MgLi(PO4)7 100%

The results of inductively coupled plasma atomic emission spectrographic element detection of the sample No. 2107192 were as follows (wt %):
Magnesium 1.11; lithium 0.35; calcium 33.1; phosphorus 21.8; zinc 1.71; iron 1.46.

The results of powder diffraction component analysis and inductively coupled plasma atomic emission spectrographic element analysis indicated that lithium, magnesium, strontium, iron, calcium and phosphorus were effectively doped.

### Embodiment 22

[Sample added with lithium magnesium zinc strontium iron calcium phosphate: No. 2107011] A composite solution containing 0.10 mol/L anhydrous calcium chloride, 0.055 mol/L magnesium sulfate heptahydrate, 0.18 mol/L lithium chloride trihydrate, 0.2 mol/L zinc acetate, 0.09 mol/L anhydrous ferric chloride, 0.18 mol/L strontium nitrate and 0.10 mol/L white granulated sugar was prepared, and 50 g of bovine cancellous bone mineral particles [0.4-1 mm, calcined at 988° C.] were added. The reaction system was baked for 300 minutes in a constant temperature box at 198° C. in a 500 mL flask, and the powder was stirred repetitively until the powder gradually become uniform black sands for further use.

The molar ratio of total cation to phosphorus in the reaction system was set as 1.3538, and 200 mL of composite solution containing 0.45 mol/L orthophosphoric acid and 0.18836 mol/L diammonium hydrogen phosphate was prepared and poured into the flask with the bone mineral particles added with calcium magnesium lithium zinc strontium iron and white granulated sugar [under the protection of a 1000 mL flask]. The reaction system was subjected to hydrothermal reaction for 24 hours at 65° C., dried by microwave medium fire and thawing fire, and then dried for 9 hours at 198° C. [stirred repetitively]. After the surface of the material was carbonized, the temperature was increased by 2.5° C. every minute up to 900° C., and this temperature was maintained for 360 minutes, then reduced to 400° C. for 180 minutes, and reduced to the room temperature 70° C. for 180 minutes, so that the sample 2107011 was obtained [62.45 g, brown]. The sample 210711 was subjected to powder diffraction and element analysis. The volume of the powdery scaffold was measured by a measuring cup, and the porosity of the powdery scaffold was calculated as about 65% according to the result of measurement.

The sample No. 2107011 was detected to obtain the following:

Ca9MgLi(PO4)7 82.0%
Ca5(PO4)3(OH) 10.4%
Ca3(PO4)2 7.6%

The results of inductively coupled plasma atomic emission spectrographic element detection of the sample No. 2107011 were as follows (wt %):
Magnesium 1.04, lithium 0.29, calcium 27.7, phosphorus 17.6, zinc 2.24, strontium 1.8, iron 0.73.

The results of powder diffraction component analysis and inductively coupled plasma atomic emission spectrographic element analysis indicated that lithium, magnesium, strontium, iron, calcium and phosphorus were effectively doped.

### Embodiment 23: Test of Scaffold Dissolution in Simulated Body Fluid

2000 mL of sodium lactate Ringer's solution was prepared from sodium lactate, sodium chloride, potassium chloride and calcium chloride, and the pH value was controlled between 6.0 and 7.0. Three parts of each of modified lithium magnesium phosphate scaffold, lithium magnesium strontium phosphate scaffold, lithium magnesium zinc phosphate and lithium magnesium iron phosphate were taken, and each part was 5 g. The modified biological bone mineral scaffold was put into the body fluid in a 200 mL medical plastic bottle at a solid-to-liquid ratio of 5 g: 100-200 mL, and the sodium lactate Ringer's solution was replaced four times a week. The replaced solution was detected by a biochemical analyzer to detect elements such as calcium, phosphorus, lithium, magnesium, potassium, sodium, strontium, zinc and iron. Four weeks later, the modified scaffold was taken out, then dried for 24 hours at 80° C., and weighed to calculate the dissolution rate. The material was subjected to powder diffraction. The results showed that the dissolution rate around the modified scaffold was 3% to 15%, indicating that the scaffold material had dissolution characteristics in the simulated body fluid.

### Embodiment 24: Test as Drug Carrier

The block scaffolds 2104041 [lithium magnesium strontium], 210423 [lithium magnesium] and 2108291 [lithium magnesium iron] each in 5 g were placed in the centers of glass dishes. Particulate scaffolds [lithium magnesium zinc iron calcium 2107192] and [lithium magnesium zinc strontium iron calcium 2107011] each in 5 g were weighed and then circularly piled in the centers of glass dishes; and, the normal saline injection was sucked with a 3 mL sucker and then dripped on the scaffold materials. The hydrophilicity of the scaffold material and its ability to contain the simulated body fluid were observed. The results showed that the scaffold materials and particulate scaffold materials had good hydrophilicity; the mass-to-volume ratio of the normal saline contained in the block scaffolds was 5 g: 10 mL, 5 g: 6 mL and 5 g: 10 mL, respectively; and, the mass-to-volume ratio of the normal saline contained in the particulate scaffolds was 5 g: 5.5 mL and 5 g: 5 mL, respectively. The block scaffolds 2104041 [lithium magnesium strontium], 210423 [lithium magnesium] and 2108291 [lithium magnesium iron] each in 5 g were weighed and placed in the centers of glass dishes. Particulate scaffolds [lithium magnesium zinc iron calcium 2107192] and [lithium magnesium zinc strontium iron calcium 2107011] each in 5 g were weighed and then circularly piled in the centers of glass dishes. Human blood plasma was sucked with a 3 mL sucker and then repeatedly dripped on the scaffolds. The results showed that the scaffold materials and particulate scaffold materials had good hydrophilicity. The scaffold and particles were uniformly stained by light yellow of the blood plasma. The mass-to-volume ratio of the human blood plasma contained in the block scaffolds was 5 g: 10 mL, 5 g: 6 mL and 5 g: 10 mL, respectively; and, the mass-to-volume ratio of the blood plasma contained in the particulate scaffolds was 5 g: 6-10 mL and 5 g: 6-10 mL, respectively. The block scaffolds 2104041 [lithium magnesium strontium], 210423 [lithium magnesium] and 2108291 [lithium magnesium iron] were cut into disks that satisfied the requirements. Particulate scaffolds [lithium magnesium zinc iron calcium 2107192] and [lithium magnesium zinc strontium iron calcium 2107011] were weighed each in 0.33 g. The scaffold materials were irrigated with the levofloxacin injection. The materials had good hydrophilicity and solution containing ability. The materials were transferred to culture dish for antibacterial test [Staphylococcus aureus, Pseudomonas aeruginosa, Escherichia coli].

### Embodiment 25: Study on Cell Adhesion on Scaffold Surface

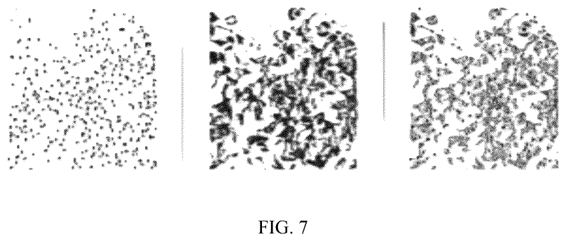

The block scaffolds 2104041 [lithium magnesium strontium], 210423 [lithium magnesium] and 2108291 [lithium magnesium iron] were selected for co-culture with MC3T3-E1. The formation of adhesion spots was observed by a confocal laser scanning microscope (CLSM). The adhesion-related protein (Vinculin) of surface cells was stained by immunofluorescence. The adhered surface cells were digested, and the expression of the adhesion-related protein (integrin, Vinculin) in cells was quantitatively analyzed by Western blot technology. The co-culture of in-vitro cells and modified scaffolds had been completed, and it was confirmed that the nano-whisker structure could promote the adhesion and spreading of cells on material surfaces and the formation of adhesion spots, which was beneficial to osteogenic expression, as shown in FIG. 7.

### Embodiment 26: In-Vivo Test as a Filling Material for Bone Defect Repair

A rabbit skull defect model [7.5 and 10 mm in diameter] was made, and the defect region was filled with a sheet-shaped modified scaffold material or bone powder with the same diameter and thickness and observed for two months and four months. The results of imageology, histology and histochemical analysis showed that the modified scaffold could well promote the repair of bone defects and had obvious osteogenic and angioblastic activities, as shown in FIG. 8.

The above description merely shows the preferred embodiments of the present invention and is not intended to limit the present invention. Although the present invention has been described in detail with reference to the above embodiments, those skilled in the art can still amend the technical solutions recorded in the above embodiments or make equivalent replacements to some technical features. Any modification, equivalent replacement and improvement made without departing from the spirit and principle of the present invention shall fall into the protection scope of the present invention.

### Embodiment 27: Mechanical Strength Detection

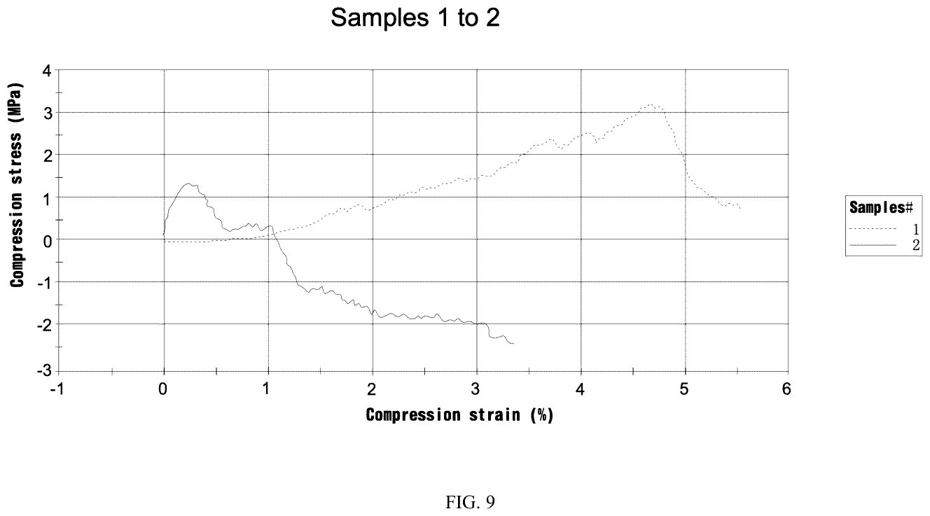

Two or three modified cylindrical scaffolds No. 2105202, 210419 and 2106281 with 1 cm in diameter and 1 cm in height and precursor bovine cancellous bone scaffolds with the same conditions were subjected to mechanical strength detection. The result of mechanical strength detection of the modified biological bone mineral scaffold No. 210520 was as follows: the average value of 1.23 MPa/cm², 1.86 MPa/cm² and 1.23 MPa/cm²=1.86 MPa/cm²; the result of mechanical strength detection of the modified biological bone mineral scaffold No. 210419 was as follows: the average value of 3.20 MPa/cm² and 1.38 MPa/cm²=2.29 MPa/cm²; the result of mechanical strength detection of the modified biological bone mineral scaffold No. 2106281 was as follows: the average value of 1.97 MPa/cm², 4.57 MPa/cm² and 1.45 MPa/cm²=2.66 MPa/cm²; and, the result of mechanical strength detection of the precursor bovine cancellous bone scaffold was as follows: the average value of 0.82 MPa/cm², 0.89 MPa/cm² and 2.19 MPa/cm²=1.30 MPa/cm². The average mechanical strength of the modified scaffolds was higher than the later. The corresponding chart data was shown in FIG. 9.

| | | | Compression | |
|---|---|---|---|---|
| | Elasticity | Maximum | strain at the | |
| | modulus(automatic | compression | maximum | |
| | Yang's) | stress | compression | Diameter |
| | (MPa ) | (MPa) | stress (%) | (mm) |
| 1 | 149.60279 | 3.20299 | 4.69317 | 12.00000 |
| 2 | 1198.93293 | 1.37501 | 0.24767 | 13.00000 |

| | |
|---|---|
| 1 | Height of compression disc (mm) |
| 2 | 14.00000 |
| 3 | 13.50000 |

What is claimed is:

1. A modified biological bone mineral scaffold doped based on lithium magnesium phosphate, which is obtained by immersing a calcined bovine or porcine cancellous bone mineral porous scaffold into a solution containing one or more of active metal ions of magnesium, lithium, strontium, zinc, iron and calcium and a phosphorus source composite solution for hydrothermal reaction, baking, drying and calcining at a high temperature.

2. The modified biological bone mineral scaffold doped based on lithium magnesium phosphate according to claim 1, wherein an alternative for the immersing a calcined bovine or porcine cancellous bone mineral porous scaffold into a solution containing one or more of active metal ions of magnesium, lithium, strontium, zinc, iron and calcium and a phosphorus source composite solution for hydrothermal reaction is: immersing the calcined bovine or porcine cancellous bone mineral porous scaffold into a metal ion source solution containing one or more of active metal ions of magnesium, lithium, strontium, zinc, iron and calcium and a white granulated sugar solution, drying off the liquid by microwave or a constant temperature box, baking and drying at 96° C. to 198° C., and then putting into the phosphorus source composite solution for hydrothermal reaction, the phosphorus source composite solution being a phosphorus source binary system.

3. The modified biological bone mineral scaffold doped based on lithium magnesium phosphate according to claim 2, wherein the hydrothermal reaction is in a constant temperature hydrothermal mode, controlled at 60° C. to 100° C. for 24 to 48 hours.

4. The modified biological bone mineral scaffold doped based on lithium magnesium phosphate according to claim 3, wherein the material-to-liquid ratio of the calcined bovine or porcine cancellous bone mineral porous scaffold to the metal ion source solution is 15-50 g: 100 mL, and the material-to-liquid ratio of the calcined bovine or porcine cancellous bone mineral porous scaffold to the phosphorus source composite solution is 15-50 g: 100 mL.

5. The modified biological bone mineral scaffold doped based on lithium magnesium phosphate according to claim 4, wherein, in the metal ion source solution, the magnesium source is one of magnesium acetate, magnesium sulfate and magnesium hydrogen phosphate; the lithium source is lithium chloride; the calcium source is one of calcium chloride and calcium hydroxide; the zinc source is one of soluble zinc salts, such as zinc nitrate or zinc acetate; the strontium source is one of soluble strontium salts, such as strontium nitrate, strontium acetate or strontium sulfate; the iron source is one of soluble iron salts, such as ferrous sulfate, ferrous chloride, ferric trichloride or ferric acetate; the phosphorus source binary system is phosphoric acid and a soluble phosphate composite solution; and, the soluble phosphate composite solution is one or a combination of diammonium hydrogen phosphate, ammonium dihydrogen phosphate, disodium hydrogen phosphate, sodium dihydrogen phosphate, dipotassium hydrogen phosphate, potassium dihydrogen phosphate and magnesium hydrogen phosphate.

6. The modified biological bone mineral scaffold doped based on lithium magnesium phosphate according to claim 5, wherein the final concentration of magnesium ions in the phosphorus source binary system containing osteogenic active metal ions is 0.05 to 0.20 mol/L; the final concentration of lithium ions in the phosphorus source binary system containing osteogenic active metal ions is 0.06 to 0.6 mol/L; the final concentration of zinc ions in the phosphorus source binary system containing osteogenic active metal ions is 0.1 to 0.6 mol/L; the final concentration of strontium ions in the phosphorus source binary system containing osteogenic active metal ions is 0.15 to 0.9 mol/L; the final concentration of ferrous ions in the phosphorus source binary system containing osteogenic active metal ions is 0.1 to 0.6 mol/L; the final concentration of calcium ions as supplement in the phosphorus source binary system containing osteogenic active metal ions is 0.15 to 1.5 mol/L; preferably, the final concentration of phosphate radials provided by phosphoric acid in the phosphorus source binary system containing osteogenic active metal ions is 0.15 to 0.9 mol/L; the final concentration of phosphate radials provided by soluble phosphate in the phosphorus source binary system containing osteogenic active metal ions is 0.06 to 0.6 mol/L; preferably, the ratio of the total molar concentration of osteogenic active cations including calcium in the reaction system to the molar concentration of phosphorus ions is 1.1-1.6:1; and, the concentration of the white granulated sugar is 0.03 to 0.2 mol/L.

7. The modified biological bone mineral scaffold doped based on lithium magnesium phosphate according to claim 1, wherein, after hydrothermal reaction, the liquid is dried off at a constant temperature, and baking and drying are performed at 75° C. to 198° C.

8. The modified biological bone mineral scaffold doped based on lithium magnesium phosphate according to claim 2, wherein, after hydrothermal reaction, the liquid is dried off at a constant temperature, and baking and drying are performed at 75° C. to 198° C.

9. The modified biological bone mineral scaffold doped based on lithium magnesium phosphate according to claim 1, wherein the high-temperature calcination is performed at 750° C. to 1200° C. for 6 to 24 hours.

10. The modified biological bone mineral scaffold doped based on lithium magnesium phosphate according to claim 2, wherein the high-temperature calcination is performed at 750° C. to 1200° C. for 6 to 24 hours.

11. The modified biological bone mineral scaffold doped based on lithium magnesium phosphate according to claim 1, wherein a method for preparing the calcined bovine or porcine cancellous bone mineral porous scaffold is as follows:
(1): strip or block-shaped calcined bovine or porcine cancellous bone mineral porous scaffold
(1-1): cutting a bovine or porcine cancellous bone into cancellous bone strips or blocks with a thickness of 0.5 to 5 cm to obtain a raw material bone, where it is also possible to prepare a cylindrical calcellous bone strip by trephination;
(1-2): stewing the raw material bone for 36 to 60 min in distilled water in a pressure cooker, then cleaning with drinking water at 50° C. to 75° C., and repeating this step for 5 to 6 times; and
(1-3): drying the raw material bone treated in the step (1-2) for 12 to 24 hours in a constant temperature box at 80° C. to 120° C., then calcining the raw material bone for 6 to 12 hours in a calcination furnace at 900° C. to 1200° C., and then slowly cooling to the room temperature to obtain calcined cancellous bone mineral porous scaffolds, the powder diffraction components of the bovine or porcine cancellous bone mineral material being hydroxyapatite;
(2): particulate bovine or porcine cancellous bone mineral porous scaffold
(2-1): cutting bovine or porcine cancellous bone into bone strips or bone blocks with a thickness of 0.5 to 6 cm to obtain a raw material bone;
(2-2): stewing the raw material bone for 36 to 60 min in distilled water in a pressure cooker, then cleaning with drinking water at 50° C. to 75° C., and repeating this step for 5 to 6 times;
(2-3): drying the raw material bone treated in the step (2-2) for 12 to 24 hours in a constant temperature box at 80° C. to 120° C., then calcining the raw material bone for 6 to 12 hours in a calcination furnace at 900° C. to 1200° C., and then slowly cooling to the room temperature to obtain calcined bovine or porcine cancellous bone mineral porous scaffolds; and
(2-4): crushing the bovine cancellous bone mineral porous scaffolds treated in the step (2-3) by a food crushing machine, and screening particulate bovine cancellous bone mineral porous scaffolds of various specifications such as 0.2-1 mm, 1-3 mm, 3-5 mm and 5-7 mm for further use by a stainless steel screen; and, crushing the porcine cancellous bone mineral porous scaffolds treated in the step (2-3) by a food crushing machine, and screening particulate porcine cancellous bone mineral porous scaffolds in 0.2-0.8 mm and 0.33-1 mm for further use by a stainless steel screen.

12. The modified biological bone mineral scaffold doped based on lithium magnesium phosphate according to claim 2, wherein a method for preparing the calcined bovine or porcine cancellous bone mineral porous scaffold is as follows:
(1): strip or block-shaped calcined bovine or porcine cancellous bone mineral porous scaffold
(1-1): cutting a bovine or porcine cancellous bone into cancellous bone strips or blocks with a thickness of 0.5 to 5 cm to obtain a raw material bone, where it is also possible to prepare a cylindrical calcellous bone strip by trephination;
(1-2): stewing the raw material bone for 36 to 60 min in distilled water in a pressure cooker, then cleaning with drinking water at 50° C. to 75° C., and repeating this step for 5 to 6 times; and
(1-3): drying the raw material bone treated in the step (1-2) for 12 to 24 hours in a constant temperature box at 80° C. to 120° C., then calcining the raw material bone for 6 to 12 hours in a calcination furnace at 900° C. to 1200° C., and then slowly cooling to the room temperature to obtain calcined cancellous bone mineral porous scaffolds, the powder diffraction components of the bovine or porcine cancellous bone mineral material being hydroxyapatite;
(2): particulate bovine or porcine cancellous bone mineral porous scaffold
(2-1): cutting bovine or porcine cancellous bone into bone strips or bone blocks with a thickness of 0.5 to 6 cm to obtain a raw material bone;
(2-2): stewing the raw material bone for 36 to 60 min in distilled water in a pressure cooker, then cleaning with drinking water at 50° C. to 75° C., and repeating this step for 5 to 6 times;
(2-3): drying the raw material bone treated in the step (2-2) for 12 to 24 hours in a constant temperature box at 80° C. to 120° C., then calcining the raw material bone for 6 to 12 hours in a calcination furnace at 900° C. to 1200° C., and then slowly cooling to the room temperature to obtain calcined bovine or porcine cancellous bone mineral porous scaffolds; and
(2-4): crushing the bovine cancellous bone mineral porous scaffolds treated in the step (2-3) by a food crushing machine, and screening particulate bovine cancellous bone mineral porous scaffolds of various specifications such as 0.2-1 mm, 1-3 mm, 3-5 mm and 5-7 mm for further use by a stainless steel screen; and, crushing the porcine cancellous bone mineral porous scaffolds treated in the step (2-3) by a food crushing machine, and screening particulate porcine cancellous bone mineral porous scaffolds in 0.2-0.8 mm and 0.33-1 mm for further use by a stainless steel screen.
